(19) United States
(12) Patent Application Publication
(10) Pub. No.: US 2020/0071743 A1
(43) Pub. Date: Mar. 5, 2020

(54) ENZYMATIC ASSAYS FOR QUANTIFYING THERAPY IN SUBJECTS WITH MUCOPOLYSACCHARIDOSIS TYPE I OR II

(71) Applicant: Sangamo Therapeutics, Inc., Richmond, CA (US)

(72) Inventors: Liching Cao, Richmond, CA (US); Yonghua Pan, Richmond, CA (US); Shelley Q. Wang, Richmond, CA (US)

(21) Appl. No.: 16/534,483

(22) Filed: Aug. 7, 2019

Related U.S. Application Data
(60) Provisional application No. 62/727,465, filed on Sep. 5, 2018.
(60) Provisional application No. 62/802,104, filed on Feb. 6, 2019.
(60) Provisional application No. 62/802,110, filed on Feb. 6, 2019.
(60) Provisional application No. 62/802,558, filed on Feb. 7, 2019.
(60) Provisional application No. 62/802,568, filed on Feb. 7, 2019.
(60) Provisional application No. 62/812,592, filed on Mar. 1, 2019.

(51) Int. Cl. C12Q 1/44

(52) U.S. Cl. CPC C12Q 1/44; G01N 2800/042; G01N 2800/52; C12Y 301/06013

(57) ABSTRACT

Described herein are enzymatic assays for assessing in vivo therapy of MPSII (Hunter) or MPSI (Hurler) subjects.

### CROSS-REFERENCE TO RELATED APPLICATIONS

The present application claims the benefit of U.S. Provisional Applications No. 62/727,465, filed Sep. 5, 2018; U.S. Provisional Application No. 62/802,104, filed Feb. 6, 2019; U.S. Provisional Application No. 62/802,110, filed Feb. 6, 2019; U.S. Provisional No. 62/802,558, filed Feb. 7, 2019; U.S. Provisional No. 62/802,568, filed Feb. 7, 2019 and U.S. Provisional No. 62/812,592, filed Mar. 1, 2019, the disclosures of which are hereby incorporated by reference in their entireties.

### TECHNICAL FIELD

The present invention concerns methods and compositions for evaluating enzyme activity, including by quantification of enzyme levels, in subjects with mucopolysaccharidosis type I (MPS I), also known as Hurler's disease, or in subjects with mucopolysaccharidosis type II (MPS II), also known as Hunter syndrome, treated in vivo with gene therapy reagents.

### BACKGROUND

Lysosomal storage diseases (LSDs) are a group of rare metabolic monogenic diseases characterized by the lack of functional individual lysosomal proteins normally involved in the breakdown of cellular waste products, including lipids, mucopolysaccharides such as glycosaminoglycans or GAGs.

MPS II is caused by mutations in the iduronate-2-sulfatase (IDS) gene which encodes an enzyme involved in the lysosomal degradation of the mucopolysaccharides glycosaminoglycans (GAG). This results in the accumulation of GAG in the urine, plasma and tissues and causes multi-systemic, progressive disease. GAGs are the most important biochemical measurement for MPS II. The accumulation of GAGs in cells and tissues, specifically dermatan sulfate and heparan sulfate, is responsible for the underlying pathology and clinical manifestation of MPS II; GAGs were the biochemical marker used by FDA and EMA to assess the pharmacodynamics of intravenous enzyme replacement therapy that is most commonly used to treat MPS II.

The only currently approved therapy for MPS II is enzyme replacement therapy (ERT). Intravenous (IV) ERT with recombinant IDS protein (idursulfase; Elaprase®, Shire) has been US FDA approved since 2006 for administration once every week in a dose of 0.5 mg/kg of body weight and has been shown to improve walking capacity in MPS II subjects 5 years and older. Limitations to ERT include the need for life-long treatment, development of neutralizing antibodies, inability of the enzyme to cross the blood brain barrier, and the inconvenience of weekly intravenous infusions. In addition, Elaprase® has a very short half-life in the plasma following treatment. When given at the approved dose (0.5 mg/kg administered weekly as a 3-hour infusion), the protein has an approximate half-life of 44 minutes (Elaprase® Solution for Intravenous Infusion Prescribing Information, Shire Human Genetics Therapies, Cambridge Mass. 2007 October). Because idursulfase cannot cross into the CNS, ERT has little to no impact on cognitive function (Parini et al. (2015) Mol Gen Metabol Rep 3:65-74). It has also been suggested to have limited efficacy for the treatment of cardiac valve disease associated with MPS II (Sato et al, ibid). In contrast to Hurler syndrome (the severe form of MPS I), hematopoietic stem cell transplantation (HSCT) has not historically been recommended for the severe form of MPS II due to a lack of efficacy in treating cognitive impairment (Guffon et al. (2009) J. Pediatric 154(5):733).

MPS I is associated with mutations in the gene encoding the iduronidase (IDUA) enzyme, which degrades glycosaminoglycans (sulfated carbohydrate polymers; GAGs). Mutations in the IDUA gene diminish or eliminate IDUA enzyme activity, which results in the accumulation of toxic GAGs in urine, plasma, and body tissues.

Many of these patients can survive into adulthood but with significant morbidity. Current therapies for MPS I include hematopoietic stem cell transplant (HSCT) and enzyme replacement therapy (ERT). If patients suffering from the severe MPS I form (MPS I-H) can be diagnosed early (<2.5 yr), therapeutic intervention by HSCT (bone marrow or umbilical cord stems cells) can prevent or reverse most clinical features including neurocognition. Currently, almost all patients with MPS I H undergo HSCT. For MPS I the mortality rate after HSCT is 15% and survival rate with successful engraftment is 56%. ERT with a polymorphic recombinant protein produced in Chinese Hamster Ovary cells, Aldurazyme® (laronidase, Sanofi Genzyme), has been in use for non-CNS therapy since 2003. This enzyme has been shown to improve pulmonary function, hepatosplenomegaly, and exercise capacity and leads to improved health related quality of life. ERT should be instituted as early as possible. Limitations to enzyme replacement therapy includes the need for life-long treatment, development of neutralizing antibodies, inability to cross the blood brain barrier, continued cardiac, orthopedic, ocular complications and the inconvenience of weekly intravenous infusions. Together, these limitations underscore the urgent need to develop a broader array of curative therapies for MPS I.

Recent studies have shown that genome-editing of liver cells in vivo in MPS I and MPS II subjects can generate the IDUA enzyme lacking in MPS I or the IDS enzyme lacking in MPS II for treatment of the disease (see, e.g., U.S. Provisional 62/802,558 and 62/802,568), thereby treating the disease. However, currently available enzymatic assays for diagnosis of MPS II (see, e.g., Voznyi et al. (2001) J. Inhert Metab Dis 24:675-680) or for assessing ERT pharmacokenetics in MPS II patients (Azadeh et al. (2017) J. Inhert Metab Dis Reports 38:89-95) do not accurately quantitate enzyme levels in gene therapy patients. In particular, the diagnostic assays are not well controlled and are not quantitative in terms of clinical parameters, such as defining the lower limit of quantification or “LLOQ”. Similarly, assays to assess ERT include the actual enzyme (provided in ERT to the subject) for use as reference, which is lacking in the gene therapy context. Moreover, ERT enzymes may behave differently from enzymes produced in vivo. See, e.g., Kim et al. (2017) J. Hum. Genetics 62-167-174. Accordingly, currently available assays for diagnosing MPS II or MPS I and evaluating ERT are not able to accurately quantify enzyme levels in MPS II or MPS I subjects treated by in vivo gene therapies.

Thus, enzymatic assays must be developed to assess in vivo treatments.

### SUMMARY

Disclosed herein are compositions and methods for assessing in vivo therapy of MPS I or II patients. The assays described herein provide a highly sensitive, quantitative, properly controlled enzyme activity assay by incorporating recombinant enzyme as an additional reference standard as well as quality control samples that span across the entire range of quantification to monitor assay performance, thereby providing a quantifiable assay to assess in vivo therapies not provided by available assays.

The methods described herein allow the enzyme curve to control and monitor the 4MU curve behavior so that the enzyme activity in the sample can be measured (assayed) consistently. Accordingly, the concentration of the enzyme in the sample can vary depending on the choice of the recombinant enzyme and results in a different back-calculated concentration. Therefore, the novel methods that provide systems using both curves (4MU and enzyme) allows for control the reaction and provides surprising and unexpectedly more accurate, sensitive, and precise quantitation of the enzyme activity as compared to current methods. In one aspect, described herein is a system or assay for assessing the levels and/or activity of IDS or IDUA in a biological system. The systems and assays involve performing multiple sample reactions alongside multiple enzyme (IDS or IDUA) reference standards, multiple substrate (label such as 4MU) reference standards and control reactions. The reference standard reactions (enzyme and substrate) are used to generate standard curved to quantify enzyme levels and/or activity in the sample reactions.

In one aspect, provided herein is a system for measuring the levels and/or activity of iduronate-2-sulfatase (IDS) in a biological sample, the system comprising the following separate reaction mixtures: (a) three or more separate reference standard reactions comprising a detectably-labeled IDS substrate, optionally 4-methylumbelliferone-alpha-L-idopayranosiduronic Acid 2-Sufate Disodium salt (4MU-IDS), and recombinant IDS (rIDS), wherein the three or more reference standard reactions include different concentrations of rIDS; (b) at least first, second and third separate quality control reactions comprising 4MU-IDS and rIDS, wherein the first quality control reaction comprises rIDS at a low quality control level, the second quality control reaction comprises rIDS at a medium quality control level and the third quality control reaction comprises rIDS at a high quality control level, optionally further comprising additional quality control reactions with rIDS at the lower and/or upper levels of quantification; (c) three or more separate substrate reactions comprising different concentrations of the detectably-labeled substrate; and (d) a plurality of sample reactions comprising the biological sample and the detectably-labeled IDS substrate, optionally wherein the separate reaction mixtures of the system are included on the same matrix such as an ELISA microplate. In certain embodiments, the system comprises duplicate reactions of at least the reference standards and quality control reactions. In certain embodiments, the biological sample comprises plasma. In other embodiments, the biological sample comprises leukocytes. Optionally, the biological sample (e.g., plasma, leukocytes) are centrifuged and/or sonicated (in any volume and/or any number of times). In certain embodiments, samples (e.g., leukocytes) are prepared by methods comprising red blood cell lysing and/or dextran treatment, preferably with sonication, optionally (but not required) with centrifugation (spinning).

In another aspect, provided herein is method of measuring the levels and/or activity of IDS in a biological sample, the method comprising the steps of: (a) providing a system of separate reaction mixtures as described herein (e.g., for IDS); (b) incubating the reactions; (c) stopping the reactions of step (b) after a period of time; (d) adding recombinant iduronidase (rIDUA) to each of the separate reactions; (e) incubating the reactions of step (d); (f) measuring the levels of detectable label from each reaction; (g) generating (i) a reference standard curve from the levels of detectable label measured in the reference standard reactions and (ii) a substrate standard curve from the levels of detectable label measured in the substrate reactions; (h) determining and/or quantifying the level and/or activity of IDS in the biological sample by measuring the levels of detectable label in the sample reactions and comparing the detected sample levels with the reference and substrate standard curves to determine enzyme activity in the sample. In certain embodiments, the reactions of step (b) are incubated for 1-3 hours and/or the reactions of step (d) are incubated for 1 to 24 hours, preferably at physiological temperature.

In another aspect, provided herein is a system for measuring the levels and/or activity of IDUA in a biological sample, the system comprising the following separate reaction mixtures: (a) three or more separate reference IDUA reactions comprising a detectably-labeled IDS substrate, optionally 4-methylumbelliferone-alpha-L-iduronide (4MU-IDUA) and recombinant IDS (rIDUA), wherein the three or more reference standard reactions include different concentrations of rIDUA; (b) three or more separate substrate reactions comprising the detectably-labeled IDUA substrate; (c) at least first, second and third separate quality control reactions comprising 4MU-IDUA and rIDUA, wherein the first quality control reaction comprises rIDUA at a low quality control level, the second quality control reaction comprises rIDUA at a mid quality control level and the third quality control reaction comprises rIDUA at a high quality control level; and (d) a plurality of sample reactions comprising the biological sample and the detectably-labeled IDUA substrate, optionally wherein the separate reaction mixtures of the system are included on the same matrix such as an ELISA microplate. In certain embodiments, the system comprises duplicate reactions of at least the reference standards and quality control reactions.

In another aspect, provided herein is a method of measuring the levels and/or activity of IDUA in a biological sample, the method comprising the steps of: (a) providing the system of separate reaction mixtures of as described herein (e.g., for IDUA); (b) incubating the reactions; (c) measuring the levels of detectable label from each reaction; (d) generating (i) a reference standard curve from the levels of detectable label measured in the reference standard reactions and (ii) a substrate standard curve from the levels of detectable label measured in the substrate reactions; and (e) determining and/or quantifying the level and/or activity of IDUA in the biological sample by measuring the levels of detectable label in the sample reactions and comparing the detected sample levels with the reference and substrate standard curves to determine enzyme activity in the sample. In certain embodiments, the reactions of step (b) are incubated for 1-3 hours, preferably at physiological temperature. In certain embodiments, the biological sample comprises plasma. In other embodiments, the biological sample comprises leukocytes. Optionally, the biological sample (e.g., plasma, leukocytes) are centrifuged and/or sonicated (in any volume and/or any number of times). In certain embodiments, samples (e.g., leukocytes) are prepared by methods comprising red blood cell lysing and/or dextran treatment, preferably with sonication, optionally (but not required) with centrifugation (spinning).

In any of the systems of methods described herein, the sample is a plasma, cellular (e.g. leukocyte) or blood sample obtained from an MPS II (IDS systems and methods) or MPS I (IDUA systems and methods) subject, optionally a subject treated with ERT and/or gene therapy reagents (e.g., nucleases that mediate integration of an IDS (MPS II) or IDUA (MPS I) transgene in vivo).

In certain embodiments of any of the systems or methods described herein, the detectably-labeled substrate is 4MU-IDS, optionally at concentrations of 0.235 μM to 50 μM in the substrate (label) reference reactions and/or in which the three or more reference standard reactions comprise dilutions (e.g., serial dilutions) of a 1.25 to 2.5 mM stock 4MU solution. In certain embodiments in the systems and methods in which an IDS standard curve is generated, the IDS standard curve covers the range of quantification from at least 0.78 to 167 nmol/hr/mL. In embodiments in which an IDUA standard curve is generated, in certain embodiments, the IDUA standard curve as described herein providing the enzyme activity covers the range of quantification from at least 0.66 to 167 nmol/hr/mL. Thus, in certain embodiments, the systems and methods (assays) described herein increase by 10-fold, 20-fold, 100-fold or more fold the ability to assess enzyme (IDS or IDUA) levels in a sample as compared to currently used assays (that do not use reference standard reactions to created an enzyme standard curve).

Further, any of the systems or methods described herein may further comprise determining an acceptable level criteria for the sample reaction measurements using one or more of the following parameters:
calculating the concentration of the standards, wherein at least 75% of the calculated concentrations for the standards must have a relative error (RE) within ±20% of low quality control (LQC), medium quality control (MQC) and high quality control (HQC);
calculating the concentration of the standards, wherein at least 75% of the calculated concentrations for the standards must have an RE within ±25% of the LLOQ or ULOQ;
substrate concentrations having a TE of ≤30% for LQC, MQC, HQC or ULOQ;
substrate concentrations having a TE of ≤40% for LLOQ;
% CVs of blank-corrected RFU for the reference and substrate standards is equal to or less than 20%; and/or
the substrate and/or reference curves have r²>0.98.

In any of the systems or methods described herein, the levels of the detectable label (e.g., 4MU) can be measured using the appropriate micro plate reader, optionally an ELISA reader in which fluorescence signal is acquired at 365 nm excitation and 450 nm emission.

These and other aspects will be readily apparent to the skilled artisan in light of disclosure as a whole.

### BRIEF DESCRIPTION OF THE DRAWINGS

The following abbreviations are used throughout:
DMSO Dimethyl Sulfoxide
RhIDUA/rIDUA Recombinant Human α-L-Iduronidase/recombinant α-L-Iduronidase
rhIDS/rIDS Recombinant Human iduronate-2-sulfatase/recombinant iduronate-2-sulfatase
BSA Bovine Serum Albumin
% CV Coefficient of variation, expressed as a percent
4MU 4-Methylumbelliferone
F/T Freeze-thaw
HQC High quality control
LLOQ Lower limit of quantification
LQC Low quality control
MQC Medium quality control
MRD Minimum Required Dilution
N/A Not applicable
NC Negative control
RE Relative error
SD Standard deviation
ULOQ Upper limit of quantification
RLU Relative light units

FIGS. 1A and 1B are schematics depicting assays for measuring IDS and IDUA activity. FIG. 1A is a schematic depicting the steps of the assay for measuring IDS activity. This is a two-step reaction requires two enzymes. In step 1, a diluted plasma sample is mixed for 3 hours at 37° C. with 4-methylumbelliferyl-α-L-idopyranosiduronic Acid 2-sulfate disodium salt (4MU-IDS), which 4MU-IDS molecule is not fluorescent in this form. IDS activity in the plasma sample removes the sulfate as shown by the solid arrow. In step 2, the IDS reaction is halted and an excess of a rIDUA enzyme is added for an overnight incubation at 37° C. to cleave the fluorescent 4MU from iduronic acid (solid arrow). IDS activity can then be interpolated from a standard curve prepared using a chemical, 4-Methylumberlliferon (4MU). Matrix background is subtracted from all samples and a log-log linear fit is used for curve fit. FIG. 1B is a schematic depicting the step of the assay for measuring IDUA activity. This is a one-step reaction requiring IDUA in which 4-MU-α-L-iduronide is cleaved by IDUA (for example in the sample) to release fluorescent 4MU. IDUA activity can then be interpolated from a standard curve prepared using a chemical, 4MU.

FIG. 2 shows a 4MU standard curve for IDS activity calculation and IDS activity of diluted samples from the same original source in separate experiments using diluted 4MU (as measured by 4MU fluorescence), generated using the previously-described methods.

FIGS. 3A through 3D show standard IDS and IDUA curves generated using assays as described herein. The left line in each curve shows assay response for each concentration of the indicated enzyme (IDS or IDUA) and the right line for each plot shows fluorescence signal for each concentration of 4MU (μM) for activity calculation. FIG. 3A shows curves of rIDS levels and the corresponding activity at lower quality control concentration (LQC, 0.3 μg/mL), middle quality control concentration (MQC, 1.25 μg/mL) and high quality control concentration (HQC, 9 μg/mL). Samples are analyzed at MRD of 1:10. FIG. 3B shows the enzyme and 4MU curves of FIG. 3A and further shows both the lower limit of quantification (LLOQ, 0.1 μg/mL) and upper limit of quantification (ULOQ, 12.5 μg/mL) as well as a summary of results including concentration interpolated from the enzyme curve (% RE=(measured-nominal)/nominal*100), mean activity (nmol/hr/mL) interpolated from 4MU and precision of measured enzyme activity expressed in % CV. Samples are analyzed at MRD of 1:10. FIG. 3C shows a standard curve generated for IDUA assays (FIG. 1B), to evaluate IDUA levels and activity at LQC (1 ng/mL), MQC (6 ng/mL) and HQC (40 ng/mL). Samples are analyzed at MRD of 1:10. FIG. 3D shows the curve of FIG. 3C and further shows both the lower limit of quantification (LLOQ, 0.39 ng/mL) and upper limit of quantification (ULOQ, 50 ng/mL) as well as a summary of results including accuracy (% RE), between run precision (% CV), within run precision (% CV) for enzyme levels (concentration (ng/mL) shown as “conc.” as shown in left standard curve labeled “IDUA”) and 4MU (μM) for activity calculation (as shown in right standard curve, labeled “4MU”).

FIGS. 4A through 4G depict results of studies conducted to determine optimum incubation time, substrate concentration, buffer preparation, and minimum required dilution (MRD). FIG. 4A shows results at the indicated incubation times. As shown, the signal increased at all concentrations of IDS from 1 to 2 to 3 hours. FIG. 4B depicts background results under the indicated conditions, where the presence of different % human plasma (“HP”) does not impact the background. The presence of different 4MU-IDS (1.25 mM vs. 2.5 mM) yields different background values, indicating 4MU-IDS contributes to assay background. Published methods (see, e.g., Voznyi et al. (2001) J. Inhert Metab Dis 24:675-680; Azadeh, ibid.) only use assay diluent to prepare 4MU standards. The results presented herein show that keeping the same % matrix and 4MU-IDS throughout and in the 4MU standard curve is important to ensure background value remains the same for all samples. The left-most bar shows background signal at 10%HP and 1.25 mM 4MU-IDS; the bar second from the left shows background signal at 20%HP and 1.25 mM 4MU-IDS; the bar second from the right shows background signal at 10%HP and 2.5 mM 4MU-IDS; and the right-most bar shows background signal at 20%HP and 2.5 mM 4MU-IDS. FIG. 4C shows the impact of proper buffer preparation. “SB” refers to substrate buffer; and “MB” refers to Mcilvaine buffer (citrate phosphate buffer). Four-fold lower assay response was observed between buffers prepared in two different commercial laboratories (“Lab 1 and Lab 2”). The left most bar shows results from assays where both the SB and MB buffer were prepared at Lab 1; the middle bar shows results when SB was prepared at Lab 2 while MB was prepared at Lab 1; the right most bar shows results when both SB and MB were prepared at Lab 1. These results demonstrate that proper SB buffer was critical for this reaction. Concentration of lead acetate is important in SB buffer and a small variation in the amount added can impact assay performance. FIG. 4D shows standard enzyme (IDS) curves generated at 5% matrix (MRD 20 indicates 1:20 matrix dilution) (top line) and 10% matrix (MRD 10 indicates 1:10 matrix dilution) (bottom line) keeping IDS concentration constant at each dilution. As shown, assay inhibition was observed with lower matrix dilution. FIG. 4E shows standard activity curve (4MU) generated in 5% matrix (MRD 20 indicates 1:20 matrix dilution) and 10% matrix (MRD 10 indicates 1:10 matrix dilution). As shown by the overlapping curves, matrix caused inhibition was not observed in the 4MU curve. FIG. 4F shows standard enzyme (IDS) curves generated at 5% matrix (MRD 20 indicates 1:20 matrix dilution) (top line) and at 10% matrix (MRD 10 indicates 1:10 matrix dilution) (bottom line) at a substrate (4MU-IDS) stock concentration of 1.25 mM. As shown, inhibition with lower sample dilution was observed at this substrate concentration. FIG. 4G shows standard enzyme (IDS) curves generated at a dilution of the sample at 5% matrix (MRD 20 indicates 1:20 matrix dilution) and at 10% matrix (MRD 10 indicates 1:10 matrix dilution) at a substrate (4MU-IDS) stock concentration of 2.5 mM. As shown by the overlapping curves, higher substrate drives the enzyme reaction and reduces inhibitory effect due to higher matrix percentage.

FIGS. 5A and 5B show dilution linearity of enzyme and activity standard curves generated using the assays described herein in which spiked samples were prepared by spiking 1000 ng/mL rIDUA in heat inactivated human plasma or 30.7 μg/mL of rIDS in heat inactivated human plasma. FIG. 5A shows IDUA (MPS I) enzyme and activity standard curves and a summary of the results. IDUA curve is shown in the left line and 4MU curve is shown in the right line. Spiked samples with rIDUA at 1000 ng/mL in human plasma was diluted to 1:50 (D50), 1:250 (D250), 1:1250 (D1250), and 1:6250 (D6250) keeping matrix constant at 10% human plasma. Dilution linearity is observed when samples are diluted within the range of quantification (D50-D6250) with % RE within ±20% and measured activity with precision (% CV)≤3.1% across all three dilutions. FIG. 5B shows similar assay performance for the IDS (MPS II) assay by spiking rIDS into heat inactivated human plasma at 30.7 μg/mL and analyzed at 1:40, 1:80, and 1:160 dilutions. Acceptance criteria: % RE±20% and % CV<20%. Dilution linearity is observed when samples are diluted within the range of quantification (1) with overall % RE at −6.08% and measured activity (nmol/hr/mL) with precision (% CV)≤2.02% across all three dilutions. As shown, the assays described herein demonstrated dilution linearity.

FIG. 6 is a graph showing selectivity and specificity of the assays described herein here. In particular, 8 of the 10 samples (circles) tested fell in the acceptable range and no signal was detected in the absence of IDS (and presence of IDUA of step 2).

FIG. 7 depicts results using IDUA assay showing no impact of hemolyzed (H) or lipemic (L) samples using the assays described herein. “BQL” refers to samples that were below the limit of quantification. As shown different dilutions for a given sample gave similar activity within assay range and no interference from hemolysis or lipemic samples was observed.

FIGS. 8A and 8B depict the stability of results obtained when samples were frozen and thawed multiple times (up to 5 times as indicated). FIG. 8A is a graph showing results from two different subjects (with differing activity levels) for IDUA enzyme assay. FIG. 8B summarized these results in tabular form for IDS enzyme assay. Relative error was calculated using 1×FT as nominal value and using formula % RE=((measured-nominal)/nomimal)*100. Acceptance criteria: % RE±20% and CV≤20%. % RE ranges from −3.43 to 1.71% and overall % CV for measured activity is ≤5.7%. As shown, assay results remained within acceptable criteria for up to 5 freeze and thaw cycles.

FIGS. 9A and 9B are graphs showing results obtained when the assay as described herein was performed on healthy donors. FIG. 9A shows IDS levels in plasma of healthy donors. FIG. 9B shows IDUA activity in plasma in healthy donors.

FIG. 10 is a graph showing that at all of LQC (bottom data points), MQC (middle data points) and HQC (top data points) for IDS assay, the assay described herein produced results in the acceptance range.

FIG. 11 shows a calibration curve generated from IDUA assays as described herein performed on leukocyte samples. See, Example 6 for further details.

### DETAILED DESCRIPTION

Disclosed herein are methods and compositions for determining IDS or IDUA activity levels in biological samples, particularly in samples obtained from subjects with MPS I (IDUA deficient) or MPS II (IDS deficient) that have been treated in vivo with ERT and/or gene therapies.

The sample (e.g., plasma) is preferably obtained from a subject with MPS II or MPS I that has been treated in vivo with reagents including a transgene for expression of IDS or IDUA, respectively, in the subject, for example nuclease-mediated integration of an IDS or IDUA transgene into a liver cell (albumin gene) of the subject such that IDS or IDUA is produced. Currently available standard assays which do not control for run variability caused by the enzyme reaction; do not accurately monitor assay performance; do not have the enzyme for use as the reference to control the range of quantitation; do not add substrate and matrix in 4MU which results in overestimating activity; do not have quantifiable range that covers both disease and healthy donor ranges, and do not define the lower limit of quantification (LLOQ), making it difficult to compare data from different laboratories and/or samples run by the same laboratory.

Thus, the assays described herein provide sensitive, quantitative assays for both MPSI and MPS II subjects treated via gene therapy or ERT and healthy subjects by controlling for run variability, accurately monitoring assay performance; defining the lower limit of quantification (LLOQ), increasing the range, accuracy, precision, dilution linearity, specificity and reproducibility of the assay, allowing for ready assessment of the subject (e.g. pre- and post-treatment).

Mucopolysaccharidosis II (MPS II), also referred to as Hunter syndrome, is an X-linked, recessive, lysosomal storage disorder found predominantly in males. The incidence of MPS II is reported as 0.3 to 0.71 per 100,000 live births (Burton & Giugliani (2012) Eur J Pediatr. (2012) April; 171(4):631-9). Applying the more conservative median life expectancy of 21.7 years for the attenuated form of the disease (the life expectancy for the severe form of the disease is 11.8 years, (Burrow et al. (2008) Biologics. June; 2(2):311-20; Young & Harper (1982) Med Genet. December; 19(6):408-11) to the yearly incidence yields an estimated prevalence of about 629 individuals with MPS II currently living in the US.

Hunter syndrome represents a disease spectrum spanning early onset, severe disease (two-thirds of subjects) with somatic and cognitive involvement, to attenuated MPS II characterized by later onset of somatic disease and little or no central nervous system (CNS) disease. The specific type of IDS mutation (>150 gene mutations have been identified) and the levels of the resulting residual IDS enzyme most likely determine the severity of disease. The residual IDS activity in the attenuated form has been measured at 0.2-2.4% of the wildtype IDS activity and those with the severe phenotype have no activity (Sukegawa-Hayasaka et al. (2006) J Inherit Metab Dis 29(6):755-61). The IDS gene is mapped to Xq28, and contains nine exons spread over 24 kb. Major deletions and rearrangements are always associated with the severe form of the disease.

Severe MPS II subjects typically start to have delayed speech and developmental delay between 18 months to 3 years of age. The disease is characterized by symptoms in severe MPS II subjects such as organomegaly, hyperactivity and aggressiveness, neurologic deterioration, joint stiffness and skeletal deformities (including abnormal spinal bones), coarse facial features with enlarged tongue, heart valve thickening, hearing loss and hernias. Joint stiffness leads to problems with walking and manual dexterity. In early childhood, subjects may display an inability to keep up with peers during physical activity, while later in life, the ability to walk even short distances may be lost and many subjects eventually become wheelchair dependent (Raluy-Callado et al. (2013) Orphanet J Rare Dis (2013) 8:101). Subjects have frequent upper respiratory infections which initially may be treated by surgical procedures such as adenotonsillectomy but ultimately may require tracheostomy and/or positive pressure ventilation (J. Ed. Wraith (2013) in Emery and Rimoin's Principles and Practice of Medical Genetics, Chapter 102.3, Rimoin, Pyeritz and Korf eds. Elsevier Ltd; Sasaki et al. (1987) Laryngoscope 97: 280-285). Major mortality factors are central nervous system involvement, cardiac involvement, and upper airway obstruction (Sato et al. (2013) Pediatr Cardiol. 34(8): 2077-2079). The life expectancy of untreated subjects with severe Hunter syndrome is into the mid teenage years with death due to neurologic deterioration and/or cardiorespiratory failure. Subjects with the attenuated form are typically diagnosed later than the severe subjects. The symptoms of the disease are similar in the severe subjects, but overall disease severity is milder with, in general, slower disease progression with no or only mild cognitive impairment. Death in the untreated attenuated form is often between the ages of 20-30 years from cardiac and respiratory disease.

Mucopolysaccharidosis type I (MPS I), also referred to as Hurler/Hurler-Scheie/Scheie syndrome, is a recessive lysosomal storage disorder. According to the National Institute of Neurological Disorders and Stroke (NINDS) factsheet for MPS I, the estimated incidence is 1 in about 100,000 births for severe MPS I, 1 in about 500,000 births for attenuated MPS I, and 1 in about 115,000 births for disease that falls between severe and attenuated.

Depending upon the specific type of IDUA mutation (more than 100 different mutations have been described) and the levels of the resulting residual IDUA enzyme, patients will develop either Hurler syndrome (MPS I H) or the attenuated variants (MPS I H/S and MPS I S). It has been estimated that 50%- 80% of all MPS I patients present with the severe form, which could be partly attributed to the relative ease of diagnosis (Muenzer et al. (2009) Pediatrics. 123(1): 19-29). MPS I H patients show symptoms of developmental delay before the end of their first year as well as halted growth and progressive mental decline between ages 2- 4 yrs. Other symptoms include organomegaly, corneal clouding, joint stiffness and skeletal deformities (including abnormal spinal bones), coarse facial features with enlarged tongue, hearing loss and hernias. The life expectancy of these MPS I H patients is less than 10 years. Patients with the attenuated form share most of these clinical manifestations but with less severe symptoms. The clinical severity of MPS I depends on the nature of the mutational changes and the degree of residual IDUA enzyme activity. Affected individuals may develop mental retardation; other central nervous system manifestations (e.g., hydrocephalus, cervical cord compression with paraplegia/quadriplegia); organomegaly; corneal clouding; joint stiffness and contractures; skeletal deformities (including abnormal spinal bones); hearing loss (deafness); hernias; chronic restrictive and obstructive pulmonary disease; and cardiac disease including arrhythmias, valve disease, coronary artery narrowing, and, rarely, cardiomyopathy and cardiac failure.

In healthy subjects, IDS enzyme is produced inside the cell and a small amount of it may leak out into the circulation due to cells' imperfect internal transport system. A steady state is established as extracellular enzyme is taken back up by receptors on the cells' surface. As a result, most of the enzyme normally produced in the body is found in the tissues, with very small concentrations of enzyme found in circulation. In contrast, ERT is an infusion directly into the bloodstream of a large bolus of enzyme designed to create high concentrations in the circulation to allow uptake into IDS- or IDUA-deficient tissues. However, ERT only produces transient high levels of IDS or IDUA enzyme, followed by rapid clearance from the circulation within a matter of minutes to hours due to the short half-life of the enzymes, and because large amounts are taken up by the liver. This limits the effectiveness of ERT because it only provides a short window of exposure of enzyme to the tissues, and within the individual cells, enzyme uptake by the cells is a slow receptor-mediated process. Thus, gene therapy (e.g., via nuclease-mediated integration of an IDS or IDUA transgene such that IDS or IDUA is produced and secreted by the liver of the subject) is an ideal therapy for MPS II or MPS I that would allow prolonged and sustained exposure of the IDS or IDUA enzyme to the tissues by producing and maintaining continuous, stable levels of enzyme in the circulation. Even low amounts of IDS or IDUA secreted continuously into the circulation could be adequate to reduce tissue GAGs and potentially provide efficacy for the compositions disclosed herein.

ERT has been shown to increase the amount of lysosomal enzyme activity in patient's leukocytes following treatment, presumably because the cells take up the enzyme from the plasma (leukocytes are lysosome-rich cells). For example, in a study of MPS I patients receiving recombinant IDUA, it was reported (see Kakkis et al (2001) NEJM 344(3)) that the mean activity of IDUA in leukocytes was 0.04 U per mg prior to treatment, and following treatment, it was measured at 4.98 U per mg seven days after infusion (i.e. immediately prior to the next treatment). Similarly, the measurement of IDS in the circulating leukocytes of MPS II patients can be useful for determining the presence of the enzyme in the plasma.

The novel highly sensitive quantitative assay described herein can be used to measure plasma IDS or IDUA activity in a subject, including healthy subjects or MPS II (IDS) or MPS II (IDUA) subjects receiving ERT and/or gene therapy. In clinical trials, the assays described herein (with a lower limit of quantification of 0.78 nmol/hour/mL) was used to measure and quantify plasma IDS activity in ERT and/or gene therapy treated patients. In clinical trials, the assays described herein (with a lower limit of quantification of 0.66 nmol/hour/mL) was used to measure and quantify plasma IDUA activity in ERT and/or gene therapy treated patients. Thus, the highly sensitive assays described herein (which exhibit 100 fold or more increased sensitivity as compared to currently used assays) greatly expanding the range of enzyme levels and/or that can be assessed in a biological sample.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (Ka) of 10⁻⁶ M⁻¹ or lower. “Affinity” refers to the strength of binding: increased binding affinity being correlated with a lower Ka.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. The term “zinc finger nuclease” includes one ZFN as well as a pair of ZFNs (the members of the pair are referred to as “left and right” or “first and second” or “pair”) that dimerize to cleave the target gene.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. Nos. 8,586,526 and 9,458,205. The term “TALEN” includes one TALEN as well as a pair of TALENs (the members of the pair are referred to as “left and right” or “first and second” or “pair”) that dimerize to cleave the target gene. Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 8,568,526; 6,140,081; 6,453,242; and 6,534,261; see also WO 98/53058; WO 98/53059; WO 98/53060; WO 02/016536 and WO 03/016496.

A “selected” zinc finger protein or TALE is a protein not found in nature whose production results primarily from an empirical process such as phage display, interaction trap or hybrid selection. See e.g., U.S. Pat. Nos. 8,586,526; 5,789,538; 5,925,523; 6,007,988; 6,013,453; 6,200,759; and WO 95/19431; WO 96/06166; WO 98/53057; WO 98/54311; WO 00/27878; WO 01/60970; WO 01/88197 and WO 02/099084.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional pairs of zinc-finger or TALEN proteins can be used for additional double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or non-coding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, U.S. Pat. Nos. 7,888,121; 7,914,796; 8,034,598 and 8,823,618, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

The “blood brain barrier” is a highly selective permeability barrier that separates the circulating blood from the brain in the central nervous system. The blood brain barrier is formed by brain endothelial cells which are connected by tight junctions in the CNS vessels that restrict the passage of blood solutes. The blood brain barrier has long been thought to prevent the uptake of large molecule therapeutics and prevent the uptake of most small molecule therapeutics (Pardridge (2005) NeuroRx 2(1): 3-14).

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of fusion molecules include, but are not limited to, fusion proteins (for example, a fusion between a protein DNA-binding domain and a cleavage domain), fusions between a polynucleotide DNA-binding domain (e.g., sgRNA) operatively associated with a cleavage domain, and fusion nucleic acids (for example, a nucleic acid encoding the fusion protein).

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP or TALEN as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

“Red Blood Cells” (RBCs) or erythrocytes are terminally differentiated cells derived from hematopoietic stem cells. They lack a nuclease and most cellular organelles. RBCs contain hemoglobin to carry oxygen from the lungs to the peripheral tissues. In fact, 33% of an individual RBC is hemoglobin. They also carry CO2 produced by cells during metabolism out of the tissues and back to the lungs for release during exhale. RBCs are produced in the bone marrow in response to blood hypoxia which is mediated by release of erythropoietin (EPO) by the kidney. EPO causes an increase in the number of proerythroblasts and shortens the time required for full RBC maturation. After approximately 120 days, since the RBC do not contain a nucleus or any other regenerative capabilities, the cells are removed from circulation by either the phagocytic activities of macrophages in the liver, spleen and lymph nodes (˜90%) or by hemolysis in the plasma (˜10%). Following macrophage engulfment, chemical components of the RBC are broken down within vacuoles of the macrophages due to the action of lysosomal enzymes.

“Secretory tissues” are those tissues in an animal that secrete products out of the individual cell into a lumen of some type which are typically derived from epithelium. Examples of secretory tissues that are localized to the gastrointestinal tract include the cells that line the gut, the pancreas, and the gallbladder. Other secretory tissues include the liver, tissues associated with the eye and mucous membranes such as salivary glands, mammary glands, the prostate gland, the pituitary gland and other members of the endocrine system. Additionally, secretory tissues include individual cells of a tissue type which are capable of secretion.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to up-regulate gene expression. When a fusion polypeptide in which a ZFP or TALE DNA-binding domain is fused to a cleavage domain, the ZFP or TALE DNA-binding domain and the cleavage domain are in operative linkage if, in the fusion polypeptide, the ZFP or TALE DNA-binding domain portion is able to bind its target site and/or its binding site, while the cleavage domain is able to cleave DNA in the vicinity of the target site.

A “functional” protein, polypeptide or nucleic acid includes any protein, polypeptide or nucleic acid that provides the same function as the wild-type protein, polypeptide or nucleic acid. A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin reistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest. A “WPRE” sequence is a woodchuck hepatitis posttranscriptional regulatory element derived from the woodchuck hepatitis virus. WPRE is a 600 bp long tripartite element containing gamma, alpha, and beta elements, in the given order (Donello et al (1992) J Virol 72:5085-5092) and contributes to the strong expression of transgenes in AAV systems (Loeb et al (1999) Hum Gene Ther 10:2295-2305). It also enhances the expression of a transgene lacking introns. In its natural form WPRE contains a partial open reading frame (ORF) for the WHV-X protein. The fully expressed WHV-X protein in the context of other viral elements like the WHV (We2) enhancer has been associated with a higher risk of hepatocarcinoma in woodchucks and mice (Hohne et. al (1990) EMBO J9(4):1137-45; Flajolet et. al (1998) J Virol 72(7):6175-80). The WHV-X protein does not appear to be directly oncogenic, but some studies suggest that under certain circumstances it can act as a weak cofactor for the generation of liver cancers associated with infection by hepadnaviruses (hepatitis B virus for man; woodchuck hepatitis virus for woodchucks). Many times, mention of “wildtype” WPRE is referring to a 591 bp sequence (nucleotides 1094-4684 in GenBank accession number J02442) containing a portion of the WHV X protein open-reading frame (ORF) in its 3′ region. In this element, there is an initial ATG start codon for WEI V-X at position 1502 and a promoter region with the sequence GCTGA at position 1488. In Zanta-Boussif (ibid), a mut6WPRE sequence was disclosed wherein the promoter sequence at position 1488 was modified to ATCAT and the start codon at position 1502 was modified to TTG, effectively prohibiting expression of WHV-X. In the J04514.1 WPRE variant, the ATG \VFW X start site is a position 1504, and a mut6 type variant can be made in the this J04514.1 strain. Another WPRE variant is the 247 bp WPRE3 variant comprising only minimal gamma and alpha elements from the wild type WPRE (Choi et al (2014) Mol Brain 7:17), which lacks the WHV X sequences.

The extracellular matrix that surrounds and binds certain types of cells is composed of numerous components, including fibrous structural proteins, such as various collagens, adhesive proteins like laminin and fibronectin, and proteoglycans that form the gel into which the fibrous structural proteins are embedded. Proteoglycans are very large macromolecules consisting of a core protein to which many long polysaccharide chains called glycosaminoglycans are covalently bound. Due to the high negative charge of the glycosaminoglycans, the proteoglycans are very highly hydrated, a property that allows the proteoglycans to form a gel-like matrix that can expand and contract. The proteoglycans are also effective lubricants. “Glycosaminoglycans” or “GAGs” are long, linear polymers of unbranched polysaccharides consisting of a repeating disaccharide unit. The repeating unit (except for keratan) consists of an amino hexose sugar (N-acetylglucosamine or N-acetylgalactosamine) along with an acidic uronic sugar (glucuronic acid or iduronic acid) or galactose. The exception to this general structure is keratan sulfate, which has galactose in place of the acidic hexose. Glycosaminoglycans are highly polar and attract water. All of the GAGs except hyaluronan are covalently linked to one of approximately 30 different core proteins to form proteoglycans. The core protein is synthesized on the rough endoplasmic reticulum and transferred to the Golgi where nucleoside diphosphate—activated acidic and amino sugars are alternately added to the nonreducing end of the growing polysaccharide by glycosyltransferases, resulting in the characteristic repeating disaccharide structure common to the GAGs. Heparin/heparan sulfate (HS GAGs) and chondroitin sulfate/dermatan sulfate (CS GAGs) are synthesized in the Golgi apparatus, where protein cores made in the rough endoplasmic reticulum are posttranslationally modified with 0-linked glycosylations by glycosyltransferases forming proteoglycans. Keratan sulfate may modify core proteins through N-linked glycosylation or 0-linked glycosylation of the proteoglycan. The fourth class of GAG, hyaluronic acid, is not synthesized by the Golgi, but rather by integral membrane synthases which immediately secrete the dynamically elongated disaccharide chain. Degradation of proteoglycans during normal turnover of the extracellular matrix begins with proteolytic cleavage of the core protein by proteases in the extracellular matrix, which then enters the cell via endocytosis. The endosomes deliver their content to the lysosomes, where the proteolytic enzymes complete the degradation of the core proteins and an array of glycosidases and sulfatases hydrolyze the GAGs to monosaccharides. The lysosomes contain both endoglycosidases, which hydrolyze the long polymers into shorter oligosaccharides, and exoglycosidases that cleave individual acidic- or amino sugars from the GAG fragments. Lysosomal catabolism of GAGs proceeds in a stepwise manner from the non-reducing end. If the terminal sugar is sulfated, then the sulfate bond must be hydrolyzed by a specific sulfatase before the sugar can be removed. When the sulfate has been removed, a specific exoglycosidase then hydrolyzes the terminal sugar from the nonreducing end of the oligosaccharide, thus leaving it 1 sugar shorter. Degradation continues in this stepwise fashion, alternating between removal of sulfates by sulfatases and cleavage of the terminal sugars by exoglycosidases. If removal of a sulfate leaves a terminal glucosamine residue, then it must first be acetylated to N-acetylglucosamine because the lysosome lacks the enzyme required to remove glucosamine. This is accomplished by an acetyltransferase that uses acetyl-CoA as the acetyl group donor. When the glucosamine residue has been N-acetylated it can be hydrolyzed by α-N-acetylglucosaminidase, allowing the continuation of the stepwise degradation of the GAG. In the case of MPS II, the terminal sugar of heparan sulfate and dermatan sulfate are sulfated, and the defective IDS enzyme is not able to remove that sulfate group. Normally, the sulfate on the terminal sugar group would be removed by iduronate-2-sulfatase (IDS) and then the GAG would be acted on by alpha iduronidase (IDUA) for removal of the terminal sugar.

The terms “subject” and “patient” are used interchangeably and refer to mammals such as human subjects and non-human primates, as well as experimental animals such as rabbits, dogs, cats, rats, mice, and other animals. Accordingly, the term “subject” or “patient” as used herein means any mammalian subject to which the altered cells of the invention and/or proteins produced by the altered cells of the invention can be administered. Subjects of the present invention include those having MPS II disorder.

Generally, the subject is eligible for treatment for MPS II. For the purposes herein, such eligible subject is one who is experiencing, has experienced, or is likely to experience, one or more signs, symptoms or other indicators of MPS II; has been diagnosed with MPS II, whether, for example, newly diagnosed, and/or is at risk for developing MPS II. One suffering from or at risk for suffering from MPS II may optionally be identified as one who has been screened for elevated levels of GAG in tissues and/or urine.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease (e.g., preventing or delaying the worsening of the disease), delay or slowing the progression of the disease, ameliorating the disease state, decreasing the dose of one or more other medications required to treat the disease, and/or increasing the quality of life.

As used herein, “delaying” or “slowing” the progression of MPS II means to prevent, defer, hinder, slow, retard, stabilize, and/or postpone development of the disease. This delay can be of varying lengths of time, depending on the history of the disease and/or individual being treated.

An “effective dose” or “effective amount” is a dose and/or amount of the composition given to a subject as disclosed herein effective to stabilize, decrease or eliminate urine GAG and/or result in measurable IDS activity in the plasma.

As used herein, “at the time of starting treatment” refers to the time period at or prior to the first exposure to a MPS II therapeutic composition such as the compositions of the invention. In some embodiments, “at the time of starting treatment” is about any of one year, nine months, six months, three months, second months, or one month prior to a MPS II drug, such as SB-913. In some embodiments, “at the time of starting treatment” is immediately prior to coincidental with the first exposure to a MPS II therapeutic composition.

The term “wheelchair dependent” means a subject that is unable to walk through injury or illness and must rely on a wheelchair to move around.

The term “mechanical ventilator” describes a device that improves the exchange of air between a subject's lungs and the atmosphere.

As used herein, “based upon” includes (1) assessing, determining, or measuring the subject characteristics as described herein (and preferably selecting a subject suitable for receiving treatment; and (2) administering the treatment(s) as described herein.

A “symptom” of MPS II is any phenomenon or departure from the normal in structure, function, or sensation, experienced by the subject and indicative of MPS II. Similarly, a “symptom” of MPS I is any phenomenon or departure from the normal in structure, function, or sensation, experienced by the subject and indicative of MPS I.

“Severe MPS II” in subjects is characterized by delayed speech and developmental delay between 18 months to 3 years of age. The disease is characterized in severe MPS II subjects by organomegaly, hyperactivity and aggressiveness, neurologic deterioration, joint stiffness and skeletal deformities (including abnormal spinal bones), coarse facial features with enlarged tongue, heart valve thickening, hearing loss and hernias. The life expectancy of untreated subjects with severe Hunter syndrome is into the mid teenage years with death due to neurologic deterioration and/or cardiorespiratory failure. “Severe MPS I” in subjects is characterized by delayed speech and developmental delay between 18 months to 3 years of age. The disease is characterized in severe MPS I subjects by organomegaly, hyperactivity and aggressiveness, neurologic deterioration, joint stiffness and skeletal deformities (including abnormal spinal bones), coarse facial features with enlarged tongue, heart valve thickening, hearing loss and hernias. “Attenuated form MPS II” or “attenuated MPS I” in subjects are typically diagnosed later than the severe subjects. The somatic clinical features are similar to the severe subjects, but overall disease severity is milder with, in general, slower disease progression with no or only mild cognitive impairment. Death in the untreated attenuated form is often between the ages of 20-30 years from cardiac and respiratory disease.

The term “supportive surgery” refers to surgical procedures that may be performed on a subject to alleviate symptoms that may be associated with a disease. For subjects with MPS II, such supportive surgeries may include heart valve replacement surgery, tonsillectomy and adenoidectomy, placement of ventilating tubes, repair of abdominal hernias, cervical decompression, treatment of carpal tunnel syndrome, surgical decompression of the median nerve, instrumented fusion (to stabilize and strengthen the spine), arthroscopy, hip or knee replacement, and correction of the lower limb axis, and tracheostomy (see Wraith et al, (2008) Eur J Pediatr. 167(3): 267-277; and Scarpa et al. (2011) Orphanet Journal of Rare Diseases, 6:72).

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal anti-inflammatory drugs (NSAIDs); ganciclovir, tacrolimus, glucocorticoids such as cortisol or aldosterone, antiinflammatory agents such as a cyclooxygenase inhibitor, a 5 -lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antagonists including anti-interferon-alpha, -beta, or -gamma antibodies, anti-tumor necrosis factor-alpha antibodies (infliximab or adalimumab), anti-TNF-alpha immunoahesin (etanercept), anti-tumor necrosis factor-beta antibodies, anti-interleukin-2 antibodies and anti-IL-2 receptor antibodies; anti-LFA-1 antibodies, including anti-CD1 la and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published 7/26/90); streptokinase; TGF-beta; streptodornase; RNA or DNA from the host; FK506; RS-61443; deoxysperguahn; rapamycin; T-cell receptor (Cohen et al., U.S. Pat. No. 5,114,721); T-cell receptor fragments (Offner et al, (1991) Science, 251: 430-432; WO90/11294; Janeway (1989), Nature, 341: 482; and WO 91/01133); and T cell receptor antibodies (EP 340,109) such as T10B9.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone), dexamethasone, glucocorticoid and betamethasone.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

A “label” is used herein to refer to information customarily included with commercial packages of pharmaceutical formulations including containers such as vials and package inserts, as well as other types of packaging.

It is to be understood that one, some, or all of the properties of the various embodiments described herein may be combined to form other embodiments of the present invention. These and other aspects of the invention will become apparent to one of skill in the art.

s Assays

### A. MPS II

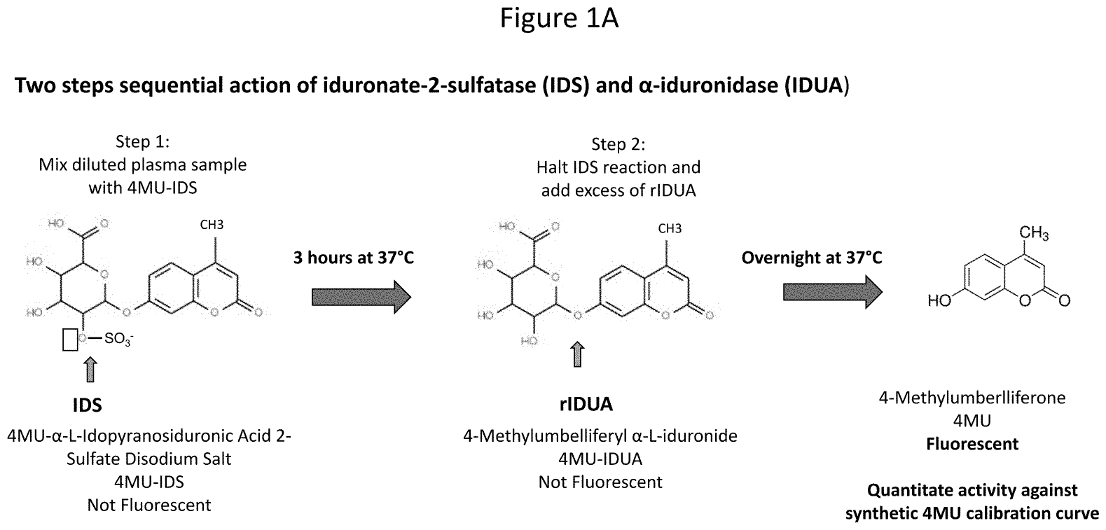

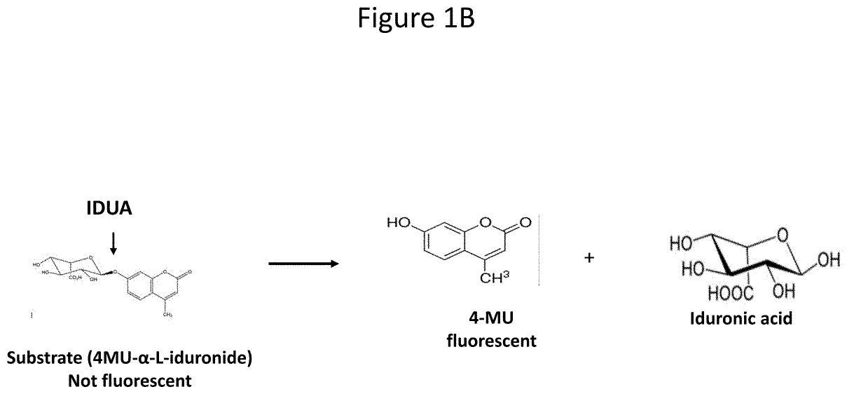

As shown in FIG. 1A, IDS enzymatic activity is measured in a two-step assay comprising (1) mixing the sample containing the IDS to be assayed with a detectably-labeled IDS substrate, typically fluorescently-labeled (e.g., 4-methylumbelliferone “4MU”) alpha-L-idopayranosiduronic Acid 2-Sufate Disodium salt (e.g., 4MU-IDS) such that the IDS present in the sample cleaves the 2′ sulfate from the 4MU-IDS: and (2) mixing exogenous lysosomal enzymes (including α-iduronidase (IDUA), but not iduronate-2-sulfatase) to remove the iduronic acid from any 4MU substrate from which the 2′ sulfate residue has already been removed by endogenous exogenous iduronate-2-sulfatase and detecting fluorescence from the free 4MU. See, e.g., FIG. 1A; Voznyi et al. (2001) J Inhert Metab Dis 24:675-680); (Azadeh et al. (2017) J. Inhert Metab Dis Reports 38:89-95).

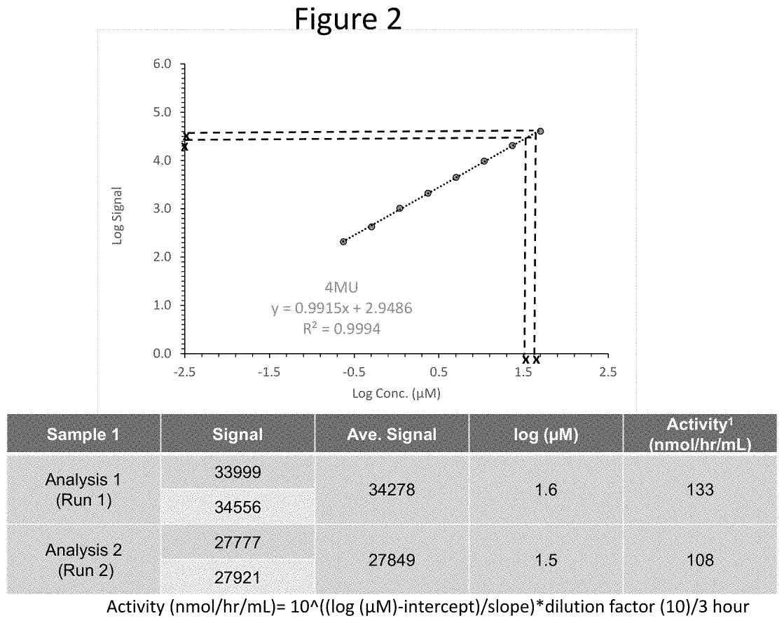

However, in currently IDS enzymatic assays, a single 4MU standard is generated by determining fluorescence from serial dilutions of this chemical. This chemical is independent of the enzyme reaction. As shown in FIG. 2, using a single 4MU standard curve can result in detection of different activity levels for the same sample and, accordingly, does not provide accurate or quantifiable results. Moreover, known assays include quality control reactions comprising pre-set values of the IDS to be assayed (e.g., the ERT formulation). See, Azadeh et al., 2017. Accordingly, because ERT formulations are different from IDS produced from a transgene as in gene therapy methods, these assays are not quantitative or accurate for patients receiving gene therapies.

The provision of both 4MU and IDS reference standards allow for quantification of clinical samples and compliance with FDA biomarker standards. Furthermore, rather than pre-set quality controls reactions that may not accurately reflect enzyme levels in patient samples, quality controls (high quality control (HQC), low quality control (LQC), mid quality control (MQC), LLOQ and/or ULOQ levels) can be generated by spiking rIDS into heat inactivated plasma and analyzed along with standard curves and/or samples as described herein for reaction monitoring. Thus, the inclusion of a rIDS reference curve (that includes quality controls) in addition to a 4MU standard curve in the assays described herein allows for detection and quantification of IDS levels in any sample, including samples obtained from healthy subjects as well as MPS II subjects receiving ERT and/or gene therapies in which IDS is produced from a transgene introduced into the subject. In addition, the novel assays and methods described herein allow for monitoring of the reaction and includes quality control for compliance with FDA acceptance levels for each patient sample. Inclusion of control reaction mixtures to generate an IDS standard curve in the assays, allows for quantitative enzyme activity assays that span across the entire range of quantification to monitor assay performance, particularly in patients receiving gene therapy (in addition to or instead of ERT).

The first and/or second reactions may be performed in any suitable reaction container. Typically, all the reactions (first and/or second controls, references, samples, etc.) are conducted at the same time, for example, on the same ELISA plate to allow for accurate quantification of each sample. Detection can be by any suitable means, including a microplate reader that can measure fluorescence at 365 nm excitation and 450 nm emission. Thus, multiple reactions are conducted at the same time, for example on an ELISA plate including duplicate wells for reference standards (rIDS-containing reactions), 4MU reference standards, duplicate sets of quality controls of HQC, MQC and LQC, and/or samples to be evaluated. Acceptable calculated values must also have % CVs of blank-corrected RFU equal to or less than 20%. Samples will be first back-calculated using the rIDS curve to ensure QC samples meet assay acceptance with at least 4 out 6 QC samples with % RE (enzyme concentration) within ±20% and no more than one sample from each level can fail. Samples are then back-calculated to 4MU standard curve and at least 4 out 6 QC samples with the ±20% mean activity range established for each level during assay qualification or validation and no more than one sample from each level can fail. Enzyme activity for each sample will be reported from the accepted run.

The 4MU standard curve (in well concentration, 0.235 μM to 50 μM) is generated as described in the art, namely by providing serial dilutions of 4MU in the same buffer composition as in enzyme reaction. Preferably, 4MU reactions are run (e.g., in duplicate) in the same assay (e.g., ELISA plate) in which the sample reactions and IDS standard curve reactions are run.

To generate the IDS standard curve (serial dilute 1.25 μg/mL of rIDS (two-fold to 0.01 μg/mL in 10% heated inactivated human plasma/assay diluent), any IDS substrate (4MU-IDS) may be used in the reactions described herein, including but not limited to a diluted or undiluted stock solution of between 1.25 to 2.5 mM (or any value therebetween). Preferably, the concentration of substrate is 2.5 mM. When diluted, the substrate may be diluted prior to addition to the reaction mixture, including but not limited diluted in buffer by 1:2, 1:3, 1:4, 1:5, 1:6, 1:7, 1:8, 1:9, 1:10, 1:20 or more. Any suitable buffer can be used for dilution, including but not limited to substrate buffer as described in the Examples. Preferably, the IDS reference reactions are run (e.g., in duplicate) in the same assay (e.g., ELISA plate) in which the sample reactions and 4MU standard curve reactions are run.

Similarly, any concentration of rIDS (data presented: 0.01 μg/mL to 1.25 μg/mL (in-well) using R&D system) may be used to generate the IDS standard curve as described herein providing the enzyme activity covers the range of quantification from 0.78 to 167 nmol/hr/mL or wider. The rIDS may be obtained from any source, including commercially available sources. Alternatively, a transgene encoding the rIDS may be introduced into a cell and the expressed protein isolated and purified from cell cultures for use in the assays.

The samples for the assay may be obtained from any tissue or part the subject, including but not limited to plasma, blood, urine, liver biopsies, CSF and the like. In certain aspects, the sample comprises plasma, which may be treated with heparin, EDTA and/or the like. Samples may be frozen prior to conducted the assay and may be freeze/thawed 1, 2, 3, 4, 5 or more times. Furthermore, the samples may be diluted prior to addition to the reaction mixture, including but not limited 1:2, 1:3, 1:4, 1:5, 1:6, 1:7, 1:8, 1:9, 1:10 or more prior to addition to the first reaction mixture. The dilution (if any) will depend on the matrix. In certain embodiments, the dilution is 1:10 or more, with a minimum dilution of 1:10. Any suitable buffer can be used for dilution, including but not limited to substrate buffer as described in the Examples. Samples may be from healthy subjects and/or MPS II subjects. Further, samples from MPS II subjects may be from treated or untreated subjects, including MPS II subjects treated with gene therapy methods (e.g., nuclease-mediated targeted integration of IDS into the liver as described below). MPS II subjects may also be receiving ERT, in which case samples are preferably collected at least 96 hours post-ERT.

The first reaction(s) (e.g., IDS standard curve, quality control reactions, patient samples) may be incubated for any amount of time, including but not limited to 1, 2, 3 or more hours. In preferred embodiments, the reaction(s) is(are) incubated for 3 hours. The first reaction is typically incubated at physiological temperature, for example 37° C. (plus or minus 5° C.). The first reaction mixture including the rIDS may include any ratio of the components (sample, rIDS, substrate, buffer, etc.).

After the selected incubation time (e.g., 3 hours), the first reaction is(are) halted, for example using any suitable quenching buffer. Any suitable quenching solution can be used, including but not limited to a citrate phosphate buffer such as Mcilvaine buffer, which may include the IDUA enzyme of the second reaction step.

During the second step, exogenous lysosomal enzymes (including α-iduronidase (IDUA), but not iduronate-2-sulfatase) to remove the iduronic acid from any 4MU substrate from which the 2′ sulfate residue has already been removed by endogenous exogenous iduronate-2-sulfatase. Recombinant IDUA (rIDUA) may be obtained from any source, including commercially available sources. Alternatively, a transgene encoding the rIDUA may be introduced into a cell and the expressed protein isolated and purified from cell cultures. Furthermore, IDUA may be added with the quenching solution or, alternatively, may be added after halting the first IDS reactions. Any concentration of rIDUA may be used, including but not limited to 1 μg/mL. The second reaction may be performed in the same container as the first reaction (e.g., in the same ELISA plate carrying one or more additional samples and/or controls) or may be performed in a different container. The second reaction(s) may be incubated for any amount of time, including but not limited to 1 to 24 (or any time therebetween) or more hours, typically overnight to 24 hours. The second reaction is typically incubated at physiological temperature, for example 37° C. (plus or minus 5° C.) and stopped before evaluation using any suitable stop buffer.

The levels of detectable moiety are measured using the appropriate micro plate reader. For ELISA plates, fluorescence signal was acquired using (365 nm excitation, 450 nm emission) plate reader.

Standard curves are generated from the reactions as described above comprising rIDS using known techniques and as shown in the appended Examples and Figures.

Therefore, each assay includes multiple reactions, for example, duplicate reactions for each of the IDS and 4MU standard curve reactions and optionally duplicate quality controls of at least three levels (e.g., LQC, MQC, HQC). Acceptable calculated values must also have % CVs of blank-corrected RFU equal to or less than 20%. Samples will be first back-calculated using rIDS curve to ensure QC samples meet assay acceptance with at least 4 out 6 QC samples with % RE (enzyme concentration) within ±20% and no more than one sample from each level can fail. Samples will then be back-calculated to 4MU standard curve and at least 4 out 6 QC samples with the ±20% mean activity range established for each level during assay qualification or validation and no more than one sample from each level can fail. Enzyme activity for each sample will be reported from the accepted run. In certain embodiments, the multiple first reactions are conducted on an ELISA plate and include: (i) duplicate IDS and 4MU standard curve reactions; (ii) HQC, MQC and/or LQC (all in duplicate) quality controls, which are back calculated from the IDS standard curve and 4MU; and (iii) subject (healthy and/or MPS II) samples.

Therefore, methods of quantifying the levels of IDS in one or more living subjects using the assays described herein are also provided in which multiple reactions, including samples from the one or more subjects; standard curve reactions and quality control reactions are conducted. Typically, the samples are run alongside two standard curves (rIDS and 4MU) and two sets of 3 quality control (HQC, MQC and LQC, in which rIDS is spiked into heat inactivated normal human plasma) reactions, each run in duplicate, for a total of 6 control reactions in addition to the sample reactions. An eight-point rIDS standard curve was prepared by 2-fold serial dilution of rIDS starting from 1.25 μg/mL to 0.01 μg/mL in assay diluent (Substrate buffer (SB) containing 0.2% BSA and 10% heat inactivated human plasma). An eight-point 4MU standard curve was prepared by 2-fold serial dilution of 4MU starting from 50 μM to 0.235 μM in assay diluent (Substrate buffer (SB) containing 0.2% BSA and 10% heat inactivated human plasma). Acceptable calculated values must also have % CVs of blank-corrected RFU equal to or less than 20%. Samples will be first back-calculated using rIDS curve to ensure QC samples meet assay acceptance with at least 4 out 6 QC samples with % RE (enzyme concentration) within ±20% and no more than one sample from each level can fail. Samples will then back-calculated to 4MU standard curve. For plate acceptance, at least 4 out 6 QC samples with the ±20% mean activity range established for each level during assay qualification or validation and no more than one sample from each level can fail. Enzyme activity for each sample will be reported. If QC samples does not meet the acceptance, the plate is rejected.

### B. MPSI

As shown in FIG. 1B, IDUA enzymatic activity is measured in a one-step assay comprising (1) mixing the sample containing the IDUA to be assayed with a detectably-labeled IDUA substrate, typically fluorescently-labeled (e.g., 4-methylumbelliferone “4MU”) 4MU-α-L-iduronide (e.g., 4MU-IDUA) such that the IDUA present in the sample cleaves the substrate to remove the iduronic acid from any 4MU substrate from which the 2′ sulfate residue has already been removed by iduronate-2-sulfatase and detecting fluorescence from the free 4MU. See, e.g., FIG. 1B.

However, in currently used IDUA enzymatic assays, a single 4MU standard is generated by determining fluorescence from serial dilutions of this chemical. This chemical is independent of the enzyme reaction. As shown in FIG. 2, using a single 4MU standard curve can result in detection of different activity levels for the same sample and, accordingly, does not provide accurate or quantifiable results.

The provision of both 4MU and IDUA reference standards in the same assay system (e.g., plate) allows for quantification of clinical samples and compliance with FDA biomarker standards. Furthermore, rather than pre-set quality controls reactions that may not accurately reflect enzyme levels in patient samples, quality controls (high quality control (HQC), low quality control (LQC), mid quality control (MQC), LLOQ and/or ULOQ levels) can be generated by spiking rIDUA into heat inactivated plasma and analyzed along with standard curves and/or samples as described herein for reaction monitoring. Thus, the inclusion of a rIDUA reference curve (that includes quality controls) in addition to a 4MU standard curve in the assays described herein allows for detection and quantification of IDUA levels in any sample, including samples obtained from healthy subjects as well as MPS I subjects receiving ERT and/or gene therapies in which IDUA is produced from a transgene (IDUA transgene) introduced into the subject. In addition, the novel assays and methods described herein allow for monitoring of the reaction and includes quality control for compliance with FDA acceptance levels for each patient sample. Inclusion of control reaction mixtures to generate an IDUA standard curve in the assays, allows for quantitative enzyme activity assays that span across the entire range of quantification to monitor assay performance, particularly in patients receiving gene therapy (in addition to or instead of ERT).

The reactions may be performed in any suitable reaction container. Typically, all the reactions (controls, references, samples, etc.) are conducted at the same time, for example, on the same ELISA plate to allow for accurate quantification of each sample. Detection can be by any suitable means, including a microplate reader that can measure fluorescence at 365 nm excitation and 450 nm emission. Thus, multiple reactions are conducted at the same time, for example on an ELISA plate including duplicate wells for reference standards (rIDUA-containing reactions), 4MU reference standards, duplicate sets of quality controls of HQC, MQC and LQC, and/or samples to be evaluated. Acceptable calculated values must also have % CVs of blank-corrected RFU equal to or less than 20%. Samples will be first back-calculated using rIDUA curve to ensure QC samples meet assay acceptance with at least 4 out 6 QC samples with % RE (enzyme concentration) within ±20% and no more than one sample from each level can fail. Samples will then back-calculated to 4MU standard curve and at least 4 out 6 QC samples with the ±20% mean activity range established for each level during assay qualification or validation and no more than one sample from each level can fail. Enzyme activity for each sample will be reported from the accepted run.

The 4MU standard curve (in well concentration is generated as described in the art, namely by providing serial dilutions of 4MU in the same buffer composition as in enzyme reaction. 4MU concentration can range to 0.235-50 μM or wider. Preferably, 4MU reactions are run (e.g., in duplicate) in the same assay (e.g., ELISA plate) in which the sample reactions and IDUA standard curve reactions are run.

To generate the IDUA standard curve (serial dilute 5 ng/mL of rIDUA two-fold to 0.039 ng/mL in 10% heated inactivated human plasma/assay diluent), any IDUA substrate (4MU-IDUA) may be used in the reactions described herein, including but not limited to a diluted or undiluted stock solution at 0.36 mM (or any value therebetween). In certain embodiments, the substrate is not diluted. When diluted, the substrate may be diluted prior to addition to the reaction mixture, including but not limited diluted in buffer by 1:2, 1:3, 1:4, 1:5, 1:6, 1:7, 1:8, 1:9, 1:10, 1:20 or more. Any suitable buffer can be used for dilution, including but not limited to substrate buffer as described in the Examples. Preferably, the IDUA reference reactions are run (e.g., in duplicate) in the same assay (e.g., ELISA plate) in which the sample reactions and 4MU standard curve reactions are run.

Similarly, any concentration of rIDUA (data presented: 0.039 ng/mL to 5 ng/mL (in-well) purchased from R&D Systems) may be used in the reactions used to generate the IDUA standard curve as described herein providing the enzyme activity covers the range of quantification from 0.66 to 223.7 nmol/hr/mL or wider. The rIDUA may be obtained from any source, including commercially available sources. Alternatively, a transgene encoding the rIDUA may be introduced into a cell and the expressed protein isolated and purified from cell cultures for use in the assays.

The samples for the assays may be obtained from any tissue or part the subject, including but not limited to plasma, blood, urine, liver biopsies, CSF and the like. In certain aspects, the sample comprises plasma, which may be treated with heparin, EDTA and/or the like. Samples may be frozen prior to conducting the assay and may be freeze/thawed 1, 2, 3, 4, 5 or more times. Furthermore, the samples may be diluted prior to addition to the reaction mixture, including but not limited 1:2, 1:3, 1:4, 1:5, 1:6, 1:7, 1:8, 1:9, 1:10 or more prior to addition to the reaction mixture. Any suitable buffer can be used for dilution, including but not limited to substrate buffer as described in the Examples. Samples may be from healthy subjects and/or MPS I subjects. Further, samples from MPS I subjects may be from treated or untreated subjects, including MPS I subjects treated with gene therapy methods (e.g., nuclease-mediated targeted integration of IDUA into the liver as described below). MPS I subjects may also be receiving ERT, in which case samples are preferably collected at least 96 hours post-ERT.

The reaction(s) (e.g., IDUA standard curve, quality control reactions, patient samples) may be incubated for any amount of time, including but not limited to 1, 2, 3 or more hours. In preferred embodiments, the reaction(s) is(are) incubated for about 3 hours. The reactions are typically incubated at physiological temperature, for example 37° C. (plus or minus 5° C.).

The levels of detectable moiety are measured using the appropriate micro plate reader. For ELISA plates, fluorescence signal was acquired using (365 nm excitation, 450 nm emission) plate reader.

Standard curves are generated from the reactions as described above comprising rIDUA using known techniques and as shown in the appended Examples and Figures.

Therefore, each assay includes multiple reactions, for example, duplicate reactions for each of the IDUA and 4MU standard curve reactions and optionally duplicate quality controls of at least three levels (e.g., LQC, MQC, HQC). In certain embodiments, the multiple first reactions are conducted on an ELISA plate and include: (i) duplicate IDUA and 4MU standard curve reactions; (ii) HQC, MQC and/or LQC (all in duplicate) quality controls, which are at levels back calculated from the IDUA standard curve and 4MU; and (iii) subject (healthy and/or MPS I) samples.

Therefore, methods of quantifying the levels of IDUA one or more living subjects using the assays described herein are also provided in which multiple reactions, including samples from the one or more subjects; standard curve reactions and quality control reactions are conducted. Typically, the samples are run alongside two standard curves (rIDUA and 4MU) and two sets of 3 quality control (HQC, MQC and LQC, in which rIDUA is spiked into heat inactivated normal human plasma) reactions, each run in duplicate), for a total of 6 control reactions in addition to the sample reactions. An eight-point rIDUA standard curve was prepared by 2-fold serial dilution of rIDUA starting from 5 ng/mL to 0.039 ng/mL in assay diluent (1×PBS containing 0.2%BSA and 10% heat inactivated human plasma). An eight-point 4MU standard curve was prepared by 2-fold serial dilution of 4MU starting from 35 μM to 0.197 μM in assay diluent (1×PBS containing 0.2% BSA and 10% heat inactivated human plasma).

Acceptable calculated values must also have % CVs of blank-corrected RFU equal to or less than 20%. Samples will be first back-calculated using rIDUA curve to ensure QC samples meet assay acceptance with at least 4 out 6 QC samples with % RE (enzyme concentration) within ±20% and no more than one sample from each level can fail. Samples will then back-calculated to 4MU standard curve. For plate acceptance, at least 4 out 6 QC samples with the ±20% mean activity range established for each level during assay qualification or validation and no more than one sample from each level can fail. Enzyme activity for each sample will be reported. If QC samples do not meet the acceptance, the plate is rejected.

### C. Qualification

For both MPS I and MPS II assays, methods of qualification and assay plate acceptance are also provided. In certain embodiments, during method qualification, both standard curves (recombinant enzyme, 4MU), accuracy (in terms of enzyme concentration) and precision (in terms of enzyme activity), dilutional linearity, sample stability, selectivity and specificity may be evaluated. Mean activity range can be established for each of the quality control samples. The established activity range for each QC level is used for assay acceptance, for example following the FDA ligand binding assay approach, used for assay plate acceptance.

Methods of evaluating assay acceptance criteria using the assays and methods described herein are also provided. In particular, data from an assay plate is acceptable when the mean back calculated concentrations for at least 75% of the standards must have RE within ±20% except at ULOQ and LLOQ with RE within ±25% and/or calibration (reference) standards have TE≤30% (except for LLOQ at ≤40%). In embodiments in which calibration standards are masked, a minimum of 6 passing calibration points must be present including LLOQ. Furthermore, the % CV of the blank-corrected relative fluorescence units (RFU) for each standard must be less than or equal to 20% and the calibration curve should have r²>0.98 is in order to be accepted.

Acceptance can also be evaluated using data from the two sets for quality controls (HQC, MQC, and LQC), run in duplicate. The mean concentration for each set of controls is back calculated from the IDS standard curve. The mean activity for each set of controls is back calculated from the 4MU standard curve. For data to be accepted, at least 4 out of the 6 (67%) controls must have % nominal values equal to ±20% of the nominal IDS concentration and the corresponding QC enzyme activity within the established activity range from method qualification for each control as follows:

QC Enzyme Activity Range (IDS)

| | Mean Activity from | Acceptable Activity Range |
|---|---|---|
| | BAL-17-080-085.02-REP | (Mean Activity ± 20%) |
| QC | nmol/mL/hr | nmol/mL/hr |
| HQC | 122 | 98-146 |
| MQC | 18.2 | 14.6-21.9 |
| LQC | 4.71 | 3.77-5.66 |

QC Enzyme Activity Range (IDUA)

| | Mean Activity from | Acceptable Activity Range |
|---|---|---|
| | BAL-17-080-083-REP | (Mean Activity ± 20%) |
| QC | nmol/mL/hr | nmol/mL/hr |
| HQC | 143 | 114-171 |
| MQC | 21.8 | 17.4-26.2 |
| LQC | 3.37 | 2.70-4.04 |

No more than one control from each level can fail the acceptance. Acceptable calculated values must also have % CVs of blank-corrected RFU equal to or less than 20%. Finally, the controls at each level must meet these criteria for acceptance.

### Nucleases

In certain embodiments, the assays described herein assess IDS or IDUA activity of an IDS or IDUA transgene integrated into a cell of the subject using one or more nucleases. Non-limiting examples of nucleases include ZFNs, TALENs, homing endonucleases, CRISPR/Cas and/or Ttago guide RNAs, that are useful for in vivo cleavage of a donor molecule carrying a transgene and nucleases for cleavage of the genome of a cell such that the transgene is integrated into the genome in a targeted manner. In certain embodiments, one or more of the nucleases are naturally occurring. In other embodiments, one or more of the nucleases are non-naturally occurring, i.e., engineered in the DNA-binding molecule (also referred to as a DNA-binding domain) and/or cleavage domain. For example, the DNA-binding domain of a naturally-occurring nuclease may be altered to bind to a selected target site (e.g., a ZFP, TALE and/or sgRNA of CRISPR/Cas that is engineered to bind to a selected target site). In other embodiments, the nuclease comprises heterologous DNA-binding and cleavage domains (e.g., zinc finger nucleases; TAL-effector domain DNA binding proteins; meganuclease DNA-binding domains with heterologous cleavage domains). In other embodiments, the nuclease comprises a system such as the CRISPR/Cas of Ttago system.

In certain embodiments, the composition and methods described herein employ a meganuclease (homing endonuclease) DNA-binding domain for binding to the donor molecule and/or binding to the region of interest in the genome of the cell. Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. Nos. 5,420,032; 6,833,252; Belfort et al. (1997) vNucleic Acids Res.25:3379-3388; Dujon et al. (1989) Gene 82:115-118; Perler et al. (1994) Nucleic Acids Res. 22, 1125-1127; Jasin (1996) Trends Genet.12:224-228; Gimble et al. (1996) J. Mol. Biol. 263:163-180; Argast et al. (1998) J. Mol. Biol. 280:345-353 and the New England Biolabs catalogue.

In certain embodiments, the methods and compositions described herein make use of a nuclease that comprises an engineered (non-naturally occurring) homing endonuclease (meganuclease). The recognition sequences of homing endonucleases and meganucleases such as I-SceI, I-CeuI, PI-PspI, PI-Sce,I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al. (1997) Nucleic Acids Res.25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 2007/0117128. The DNA-binding domains of the homing endonucleases and meganucleases may be altered in the context of the nuclease as a whole (i.e., such that the nuclease includes the cognate cleavage domain) or may be fused to a heterologous cleavage domain.

In other embodiments, the DNA-binding domain of one or more of the nucleases used in the methods and compositions described herein comprises a naturally occurring or engineered (non-naturally occurring) TAL effector DNA binding domain. See, e.g., U.S. Pat. No. 8,586,526, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like (TAL) effectors which mimic plant transcriptional activators and manipulate the plant transcriptome (see Kay et al. (2007) Science 318:648-651). These proteins contain a DNA binding domain and a transcriptional activation domain. One of the most well characterized TAL-effectors is AvrBs3 from Xanthomonas campestgris pv. Vesicatoria (see Bonas et al. (1989) Mol Gen Genet 218: 127-136 and WO2010079430). TAL-effectors contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al. (2006) J Plant Physiol 163(3): 256-272). In addition, in the phytopathogenic bacteria Ralstonia solanacearum two genes, designated brgll and hpx17 have been found that are homologous to the AvrBs3 family of Xanthomonas in the R. solanacearum biovar 1 strain GMI1000 and in the biovar 4 strain RS1000 (See Heuer et al. (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas. See, e.g., U.S. Pat. No. 8,586,526, incorporated by reference in its entirety herein.

Specificity of these TAL effectors depends on the sequences found in the tandem repeats. The repeated sequence comprises approximately 102 bp and the repeats are typically 91-100% homologous with each other (Bonas et al, ibid). Polymorphism of the repeats is usually located at positions 12 and 13 and there appears to be a one-to-one correspondence between the identity of the hypervariable diresidues (RVDs) at positions 12 and 13 with the identity of the contiguous nucleotides in the TAL-effector's target sequence (see Moscou and Bogdanove, (2009) Science 326:1501 and Boch et al. (2009) Science 326:1509-1512). Experimentally, the natural code for DNA recognition of these TAL-effectors has been determined such that an HD sequence at positions 12 and 13 leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al, ibid). Engineered TAL proteins have been linked to a FokI cleavage half domain to yield a TAL effector domain nuclease fusion (TALEN) exhibiting activity in a yeast reporter assay (plasmid based target). See, e.g., U.S. Pat. No. 8,586,526; Christian et al. (2010) Genetics epub 10.1534/genetics.110.120717).

In certain embodiments, the DNA binding domain of one or more of the nucleases used for in vivo cleavage and/or targeted cleavage of the genome of a cell comprises a zinc finger protein. Preferably, the zinc finger protein is non-naturally occurring in that it is engineered to bind to a target site of choice. See, for example, See, for example, Beerli et al. (2002) Nature Biotechnol.20:135-141; Pabo et al. (2001) Ann. Rev. Biochem.70:313-340; Isalan et al. (2001) Nature Biotechnol.19:656-660; Segal et al. (2001) Curr. Opin. Biotechnol.12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; and 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197. In addition, enhancement of binding specificity for zinc finger binding domains has been described, for example, in co-owned International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 8,772,453; 6,479,626; 6,903,185; and 7,153,949 for exemplary linker sequences. The proteins described herein may include any combination of suitable linkers between the individual zinc fingers of the protein.

Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536 and WO 03/016496.

In certain embodiments the DNA-binding domains bind to albumin, e.g., DNA-binding domains of the ZFPs designated SBS-47171 and SBS-47898 or the ZFPs designated SBS-71557 and SBS-71728. See, e.g., U.S. Patent Publication No. 2015/0159172 and U.S. Ser. No. 16/271,250. The MPS II patients may be treated in any way, including but not limited to as described in 62/802,558 and 62/802,568 with AAV formulations encoding left and right ZFNs separately (e.g., SBS-47171 and SB S-47898 separately of SBS-71557 and SBS-71728 separately) and an hIDS transgene (for MPS II subjects) or an hIDUA transgene (for MPS I subjects).

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

In certain embodiments, the DNA-binding domain of the nuclease is part of a CRISPR/Cas nuclease system, including, for example a single guide RNA (sgRNA). See, e.g., U.S. Pat. No. 8,697,359 and U.S. Patent Publication No. 2015/0056705. The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein. Additional non-limiting examples of RNA guided nucleases that may be used in addition to and/or instead of Cas proteins include Class 2 CRISPR proteins such as Cpfl. See, e.g., Zetsche et al. (2015) Cell 163:1-13.

In some embodiments, the DNA binding domain is part of a TtAgo system (see Swarts et al, ibid; Sheng et al, ibid). In eukaryotes, gene silencing is mediated by the Argonaute (Ago) family of proteins. In this paradigm, Ago is bound to small (19-31 nt) RNAs. This protein-RNA silencing complex recognizes target RNAs via Watson-Crick base pairing between the small RNA and the target and endonucleolytically cleaves the target RNA (Vogel (2014) Science 344:972-973). In contrast, prokaryotic Ago proteins bind to small single-stranded DNA fragments and likely function to detect and remove foreign (often viral) DNA (Yuan et al., (2005) Mol. Cell 19, 405; Olovnikov, et al. (2013) Mol. Cell 51, 594; Swarts et al., ibid). Exemplary prokaryotic Ago proteins include those from Aquifex aeolicus, Rhodobacter sphaeroides, and Thermus thermophilus.

One of the most well-characterized prokaryotic Ago protein is the one from T. thermophilus (TtAgo; Swarts et al. ibid). TtAgo associates with either 15 nt or 13-25 nt single-stranded DNA fragments with 5′ phosphate groups. This “guide DNA” bound by TtAgo serves to direct the protein-DNA complex to bind a Watson-Crick complementary DNA sequence in a third-party molecule of DNA. Once the sequence information in these guide DNAs has allowed identification of the target DNA, the TtAgo-guide DNA complex cleaves the target DNA. Such a mechanism is also supported by the structure of the TtAgo-guide DNA complex while bound to its target DNA (G. Sheng et al., ibid). Ago from Rhodobacter sphaeroides (RsAgo) has similar properties (Olivnikov et al. ibid).

Exogenous guide DNAs of arbitrary DNA sequence can be loaded onto the TtAgo protein (Swarts et al. ibid.). Since the specificity of TtAgo cleavage is directed by the guide DNA, a TtAgo-DNA complex formed with an exogenous, investigator-specified guide DNA will therefore direct TtAgo target DNA cleavage to a complementary investigator-specified target DNA. In this way, one may create a targeted double-strand break in DNA. Use of the TtAgo-guide DNA system (or orthologous Ago-guide DNA systems from other organisms) allows for targeted cleavage of genomic DNA within cells. Such cleavage can be either single- or double-stranded. For cleavage of mammalian genomic DNA, it would be preferable to use of a version of TtAgo codon optimized for expression in mammalian cells. Further, it might be preferable to treat cells with a TtAgo-DNA complex formed in vitro where the TtAgo protein is fused to a cell-penetrating peptide. Further, it might be preferable to use a version of the TtAgo protein that has been altered via mutagenesis to have improved activity at 37 degrees Celsius. TtAgo-RNA-mediated DNA cleavage could be used to effect a panopoly of outcomes including gene knock-out, targeted gene addition, gene correction, targeted gene deletion using techniques standard in the art for exploitation of DNA breaks.

Thus, the nuclease comprises a DNA-binding domain in that specifically binds to a target site in any gene into which it is desired to insert a donor (transgene).

### B. Cleavage Domains

Any suitable cleavage domain can be associated with (e.g., operatively linked) to a DNA-binding domain to form a nuclease. For example, ZFP DNA-binding domains have been fused to nuclease domains to create ZFNs—a functional entity that is able to recognize its intended nucleic acid target through its engineered (ZFP) DNA binding domain and cause the DNA to be cut near the ZFP binding site via the nuclease activity. See, e.g., Kim et al. (1996) Proc Natl Acad Sci USA 93(3):1156-1160. More recently, ZFNs have been used for genome modification in a variety of organisms. See, for example, U.S. Patent Publication Nos. 2003/0232410; 2005/0208489; 2005/0026157; 2005/0064474; 2006/0188987; 2006/0063231; and International Publication WO 07/014275. Likewise, TALE DNA-binding domains have been fused to nuclease domains to create TALENs. See, e.g., U.S. Pat. No. 8,586,526. CRISPR/Cas nuclease systems comprising single guide RNAs (sgRNAs) that bind to DNA and associate with cleavage domains (e.g., Cas domains) to induce targeted cleavage have also been described. See, e.g., U.S. Pat. Nos. 8,697,359 and 8,932,814 and U.S. Patent Publication No. 2015/0056705.

As noted above, the cleavage domain may be heterologous to the DNA-binding domain, for example a zinc finger DNA-binding domain and a cleavage domain from a nuclease or a TALEN DNA-binding domain and a cleavage domain from a nuclease; a sgRNA DNA-binding domain and a cleavage domain from a nuclease (CRISPR/Cas); and/or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res.25:3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press,1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However, any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a zinc finger binding domain and two Fok I cleavage half-domains can also be used. Parameters for targeted cleavage and targeted sequence alteration using zinc finger-Fok I fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in U.S. Pat. No. 7,888,121, incorporated herein in its entirety. Additional restriction enzymes also contain separable binding and cleavage domains, and these are contemplated by the present disclosure. See, for example, Roberts et al. (2003) Nucleic Acids Res.31:418-420.

In certain embodiments, the cleavage domain comprises one or more engineered cleavage half-domain (also referred to as dimerization domain mutants) that minimize or prevent homodimerization, as described, for example, in U.S. Pat. Nos. 8,772,453; 8,623,618; 8,409,861; 8,034,598; 7,914,796; and 7,888,121, the disclosures of all of which are incorporated by reference in their entireties herein. Amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains.

Exemplary engineered cleavage half-domains of FokI that form obligate heterodimers include a pair in which a first cleavage half-domain includes mutations at amino acid residues at positions 490 and 538 of FokI and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. U.S. Pat. Nos. 7,914,796 and 8,034,598, the disclosures of which are incorporated by reference in their entireties. In certain embodiments, the engineered cleavage half-domain comprises mutations at positions 486, 499 and 496 (numbered relative to wild-type FokI), for instance mutations that replace the wild type Gln (Q) residue at position 486 with a Glu(E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). See, e.g., U.S. Pat. No. 8,772,453. In other embodiments, the engineered cleavage half domain comprises the “Sharkey” and/or “Sharkey” mutations (see Guo et al, (2010) J. Mol. Biol. 400(1):96-107).

Engineered cleavage half-domains described herein can be prepared using any suitable method, for example, by site-directed mutagenesis of wild-type cleavage half-domains (FokI) as described in U.S. Pat. Nos. 7,888,121; 7,914,796; 8,034,598; and 8,623,618.

Alternatively, nucleases may be assembled in vivo at the nucleic acid target site using so-called “split-enzyme” technology (see, e.g. U.S. Patent Publication No. 2009/0068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in U.S. Pat. No. 8,563,314. Expression of the nuclease may be under the control of a constitutive promoter or an inducible promoter, for example the galactokinase promoter which is activated (de-repressed) in the presence of raffinose and/or galactose and repressed in presence of glucose.

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong et al, (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek ibid and Cong, ibid).

The nuclease(s) as described herein may make one or more double-stranded and/or single-stranded cuts in the target site. In certain embodiments, the nuclease comprises a catalytically inactive cleavage domain (e.g., FokI and/or Cas protein). See, e.g., U.S. Pat. Nos. 9,200,266; 8,703,489 and Guillinger et al. (2014) Nature Biotech. 32(6):577-582. The catalytically inactive cleavage domain may, in combination with a catalytically active domain act as a nickase to make a single-stranded cut. Therefore, two nickases can be used in combination to make a double-stranded cut in a specific region. Additional nickases are also known in the art, for example, McCaffery et al. (2016) Nucleic Acids Res. 44(2):e11. doi: 10.1093/nar/gkv878. Epub 2015 Oct. 19.

Thus, any nuclease comprising a DNA-binding domain and cleavage domain can be used. In certain embodiments, the nuclease comprises a ZFN made up of left and right ZFNs, for example a ZFN comprising a first ZFN comprising a ZFP designated SBS-47171 or SBS-and a cleavage domain and a second ZFN comprising a ZFP designated SBS-47898 and a cleavage domain. In certain embodiments, the left and right (first and second) ZFNs of the ZFN are carried on the same vector and in other embodiments, the paired components of the ZFN are carried on different vectors, for example two AAV vectors, one designated SB-47171 AAV (an AAV2/6 vector carrying ZFN comprising the ZFP designated SBS-47171) and the other designated SB-47898 AAV (an AAV2/6 vector carrying ZFN comprising the ZFP designated SB S-47898).

### Target Sites

As described in detail above, DNA domains can be engineered to bind to any sequence of choice in a locus, for example an albumin or other safe-harbor gene. An engineered DNA-binding domain can have a novel binding specificity, compared to a naturally-occurring DNA-binding domain. Engineering methods include, but are not limited to, rational design and various types of selection. Rational design includes, for example, using databases comprising triplet (or quadruplet) nucleotide sequences and individual (e.g., zinc finger) amino acid sequences, in which each triplet or quadruplet nucleotide sequence is associated with one or more amino acid sequences of DNA binding domain which bind the particular triplet or quadruplet sequence. See, for example, co-owned U.S. Pat. Nos. 6,453,242 and 6,534,261, incorporated by reference herein in their entireties. Rational design of TAL-effector domains can also be performed. See, e.g., U.S. Patent Publication No. 2011/0301073.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237.

Selection of target sites; nucleases and methods for design and construction of fusion proteins (and polynucleotides encoding same) are known to those of skill in the art and described in detail in U.S. Patent Publication Nos. 2005/0064474 and 2006/0188987, incorporated by reference in their entireties herein.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences 6 or more amino acids in length. The proteins described herein may include any combination of suitable linkers between the individual DNA-binding domains of the protein. See, also, U.S. Pat. No. 8,586,526.

In certain embodiments, the target site(s) for the DNA-binding domain(s) (is)are within an albumin gene. See, e.g., U.S. Patent Publication No. 2015/0159172.

### Assays

As noted above, insertion of an exogenous sequence (also called a “donor sequence” or “donor”), for example for correction of a mutant gene or for increased expression of a gene encoding a protein lacking or deficient in MPS II disease (e.g., IDS) or MPS I (IDUA) is provided.

The assays described herein allow for sensitive quantification of IDS or IDUA activity levels in the plasma of a subject treated with the methods and compositions disclosed herein.

In certain embodiments, the donor vector is a vector as shown in SB-IDS AAV or as shown SB-IDUA-AAV.

### EXAMPLES

### Example 1: Overview of Iduronate-2-sulfatase Enzyme Assay

An improved plasma IDS activity assay was developed as follows. Iduronate-2-sulfatase is a lysosomal enzyme that removes a sulfate residue from the 2′ position of an iduronic acid residue that is present in both heparan sulfate and dermatan sulfate. This assay used an artificial 4MU substrate that contained a terminal iduronic acid. However, in order for the fluorescence of 4MU to be released, the entire iduronic acid moiety must be removed from the substrate. The removal of iduronic acid was catalyzed by the α-iduronidase enzyme, and this can only occur after the removal of the sulfate residue by iduronate-2-sulfatase. Therefore, this assay was a two-step reaction. See, also, FIG. 1A. During the first step, endogenous iduronate-2-sulfatase was given the opportunity to cleave the 2′ sulfate residue from the iduronic acid residue at the end of the 4MU substrate. During the second step, exogenous lysosomal enzymes (including α-iduronidase, but not iduronate-2-sulfatase) were added to the reaction. The α-iduronidase enzyme can remove the iduronic acid from any 4MU substrate from which the 2′ sulfate residue has already been removed by endogenous iduronate-2-sulfatase. The removal of the terminal iduronic acid from the 4MU substrate releases its fluorescence, which is observed using a fluorometer. However, if no endogenous iduronate-2-sulfatase enzyme is present within the patient sample, the 2′ sulfate residue cannot be removed, which prevents the entire iduronic acid moiety from being removed, thereby quenching the fluorescence of the 4MU substrate (Voznyi et al (2001) J Inher Metab Dis 24: 675-680; Azadeh et al. 2017, ibid.).

The novel assays described herein include additional reagents, including recombinant IDS reference standard (in step 1) and/or additional controls (e.g., quality control samples), addition of all reaction components to 4MU to uniform background fluoresce signal, to provide a quantitative enzyme activity that spans across the entire range of quantification and in which assay performance can be monitored.

### Example 2: Generation of Standard Curves

In known assays, a standard curve for either an IDS or IDUA assay is generated by diluting 4MU for activity calculations. Moreover, the reaction is not monitored. As shown in FIG. 2, these assays of the same sample provide different results depending on the reaction. Therefore, standard curves generated by known assays are unsuitable for assessing in vivo therapies.

Accordingly, in the IDS assays described herein, rIDS was introduced into the first step of the assay to monitor assay performance and to define a quantifiable range. In particular, plasma from subjects was separated via centrifugation from whole blood (heparinized, or EDTA preserved). Plasma was separated from whole blood via centrifugation. After centrifugation, the top, liquid layer (plasma) was carefully pulled or poured off and collected in a separate, appropriate collection tube. This tube containing plasma is frozen and sent packed in dry ice.

Frozen plasma samples were removed from the freezer and thawed quickly at 37° C. water bath prior to dilution. Plasma samples were diluted 1:10 with substrate buffer (10 μL plasma+90 μL substrate buffer) in a separate microcentrifuge tube. In each patient/control tube, 10 μL diluted plasma+20 μL 2.5 mM Hunter substrate 4MU-IDS were combined in a microplate and incubated in a 37° C. incubator for 3 hours. 50 μL Quenching solution (2×Mcilvaine buffer with 0.2% BSA and 1 μg/mL recombinant human α-L-Iduronidase) was added to each sample and the reaction plate was put back in the 37° C. incubator for 24 hours. 40 μL of each reaction was transferred to a flat white opaque plate and 100 μL stop buffer was added. Fluorescence signal was acquired using (365 nm excitation, 450 nm emission) plate reader. Total enzyme activity was determined using the following calculations:

Plasma: Average corrected reading x dilution factor (10)=nmoles of substrate hydrolyzed per 3 hours per mL plasma. Normal plasma values were from 82-200 nmol/hr/mL (determined from 50 donors). The lower limit of quantification (LLOQ) of enzyme activity was 0.78 nmol/mL/hr. The upper limit of the analytical measurement range for enzyme activity was 167 nmol/mL/hr.

Substrate buffer was prepared as follows: 0.1 M sodium acetate and was combined with 0.01M lead acetate and adjust to pH of 5.0 using glacial acetic acid. 0.2% BSA was added to substrate buffer on the day of use for sample dilution. Hunter substrate 4MU-aIdoA-2S also referred as 4MU-IDS (2.5 mM) was purchased commercial.

Quenching solution: 2×Mcilvaine buffer was prepared at 0.4M sodium-phosphate dibasic and 0.2M citrate, pH 4.5. 0.2% BSA was added to 2xMcilvaine buffer on the day of use. Quenching solution was prepared by diluting recombinant human α-L-Iduronidase (R&D system) in 2×Mcilvaine buffer containing 0.2% BSA at final concentration of 1 μg/mL.

This assay has a lower limit of quantitation of 0.78 nmol/mL/hr. Reference ranges (nmol/mL/hr) for unaffected individuals is 82-200, while baseline for MPS II patients (>96h post-ERT) is estimated at 0-10.

For IDUA assays (MPS I), IDUA standard curves were generated as described above using a 4MU-IDUA substrate in a single-step reaction as shown in FIG. 1B. Details of the IDUA assay conditions are provided in Example 6 below.

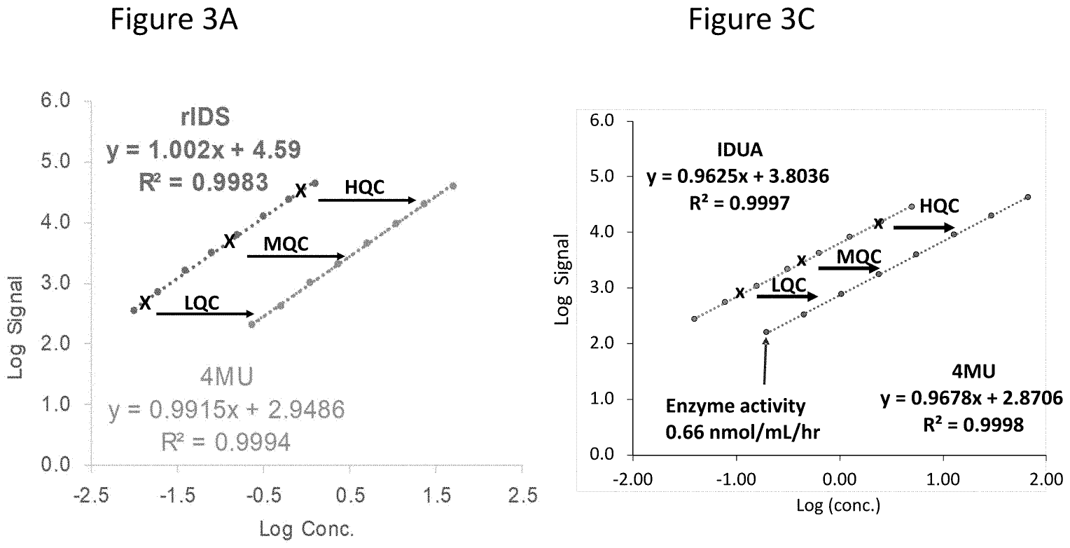

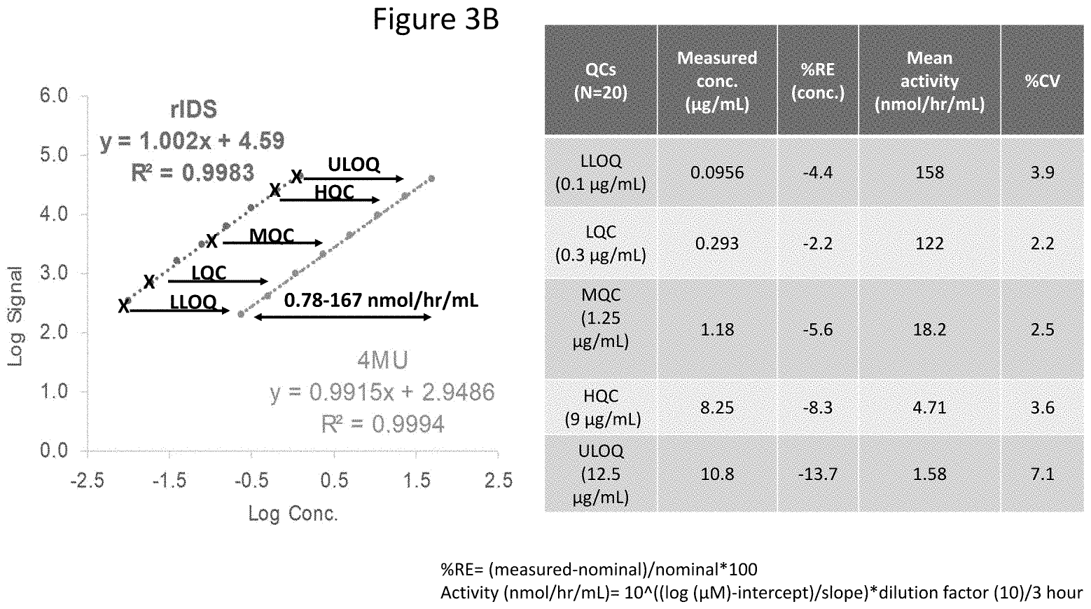

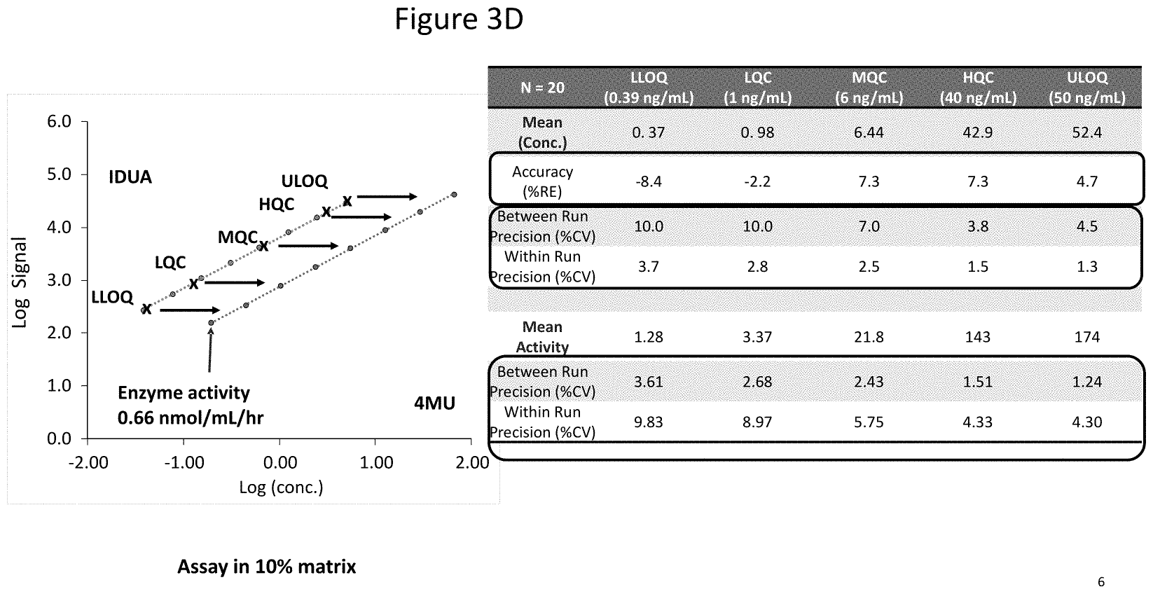

As shown in FIG. 3A through FIG. 3D, the curves generated for IDS (FIGS. 3A and 3B) and IDUA (FIGS. 3C and 3D) covered the range of quantification and conformed to quantitative biomarker assays ligand binding assay acceptance guidelines. Five levels of quality control samples were used in method qualification to ensure the assay is accurate and precise and to define the range of quantification of the enzyme assay.

### Example 3: Improvement of Assay Conditions

Assay conditions were assessed to optimize incubation times, minimize background, buffer conditions, substrate (4MU-IDS) concentration and minimum required dilution (MRD).

### A. Incubation Time

IDS assays were conducted as described above in Example 2 using 0.010 to 1.25 μg/mL of rIDS and incubating the reactions for 1, 2 or 3 hours.

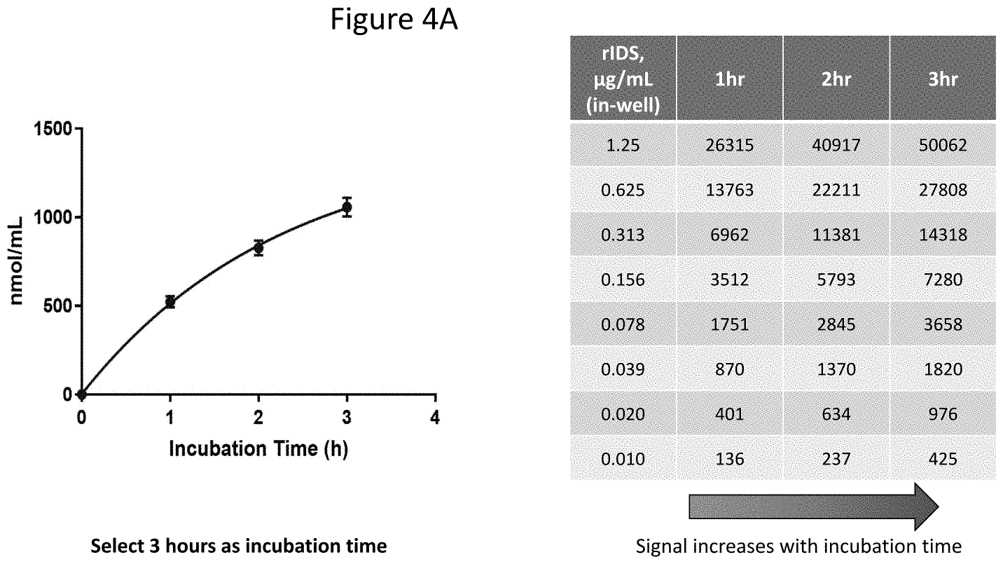

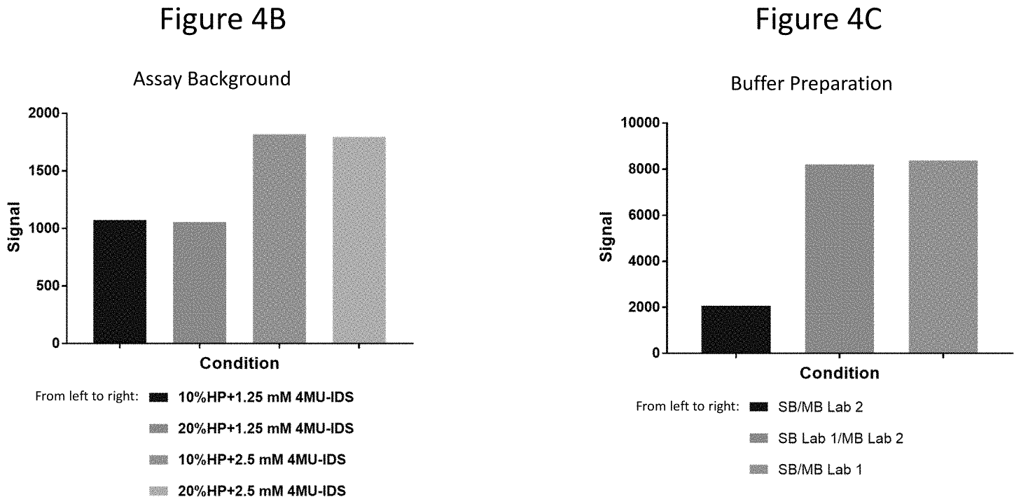

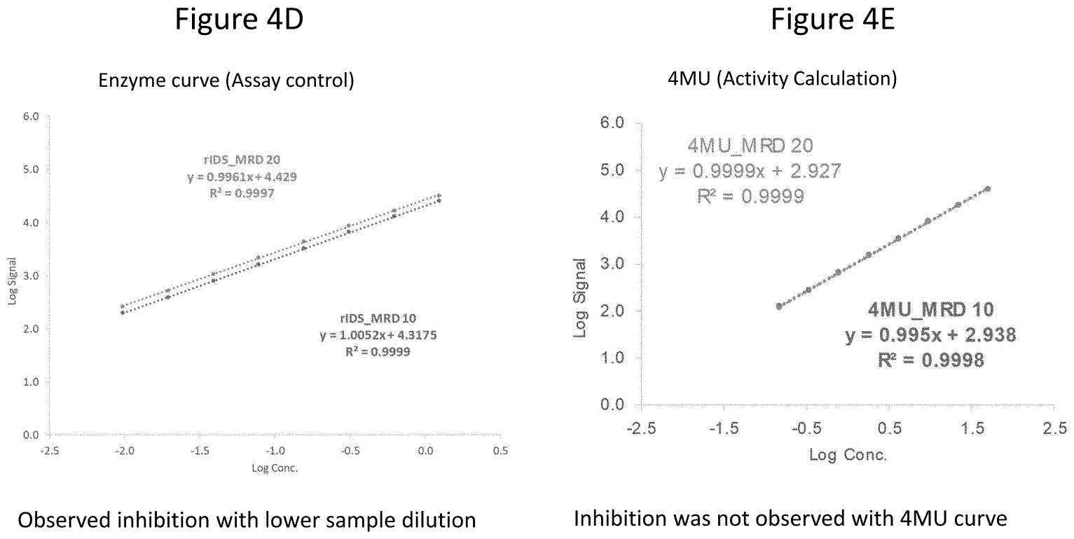

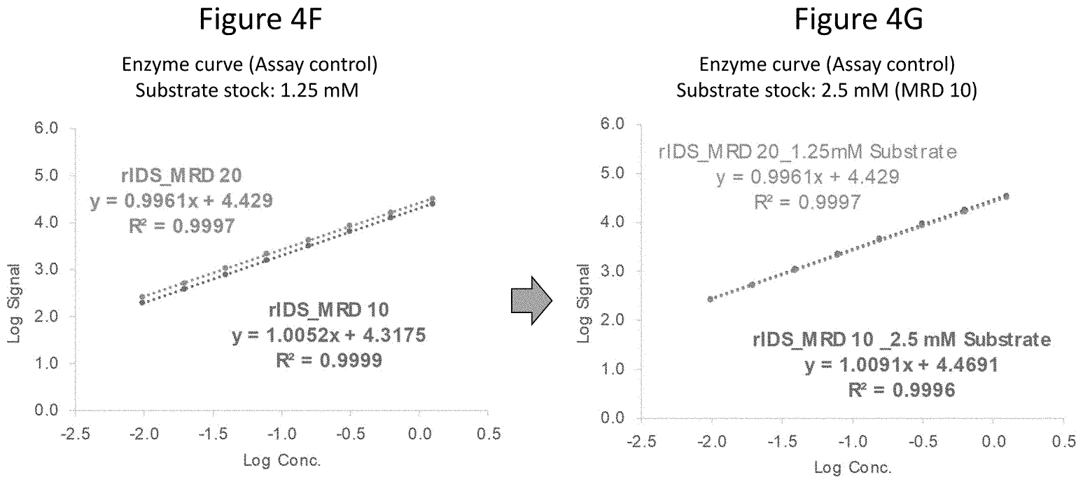

As shown in FIG. 4A, the signal detected increased at all concentrations of rIDS at the longest incubation time of 3 hours.

Accordingly, 3 hours was selected for the incubation time of step 1 (FIG. 1A).

### B. Background Levels

IDS assays were conducted as described above in Example 2 using 1.25 mM or 2.5 mM of the 4MU-IDS substrate (each at either 10% HP or 5% HP).

As shown in FIG. 4B, background levels were lower when 1.25 mM of the substrate was used in the assay as compared to 2.5 mM substrate.

### C. Buffer Preparation

IDS assays were also conducted as described in Example 2 to assess buffer preparation using different preparations of substrate buffer (“SB”) and citrate phosphate Mcilvaine buffer (“MB”).

As shown in FIG. 4C, proper preparation of buffers (see Example 2 above) is critical to maximizing the signal obtained.

### D. Minimum Required Dilution

IDS assays were also conducted as described above to assess MRD using either sample dilutions in 5% matrix (MRD of 1:20) and 10% matrix (MRD of 1:10) of either the rIDS reference standard or 4MU-IDS substrate.

As shown in FIGS. 4D and 4E, no inhibition was observed following dilution of the substrate 4MU (FIG. 4E). However, for the rIDS enzyme, inhibitor was observed at the lower sample dilution (FIG. 4D).

### E. Substrate Concentration

IDS assays were also conducted as described above to assess the impact of 4MU-IDS substrate concentration using either a stock concentration of 1.25 mM or 2.5 mM at sample dilutions in 5% matrix (MRD of 1:20) and 10% matrix (MRD of 1:10) (See, part D above).

As shown in FIGS. 4F and 4G, inhibition of the rIDS curve was observed at lower substrate concentration at the lower dilution (FIG. 4F). However, at the higher substrate concentration, lower dilution sample yielded comparable signal as higher dilution at lower substrate concentration. Higher concentration of substrate and lower sample dilution can improve assay sensitivity. (FIG. 4G).

### Example 4: Method Qualification

Having established the accuracy and precision (see, Example 2 and FIG. 3) and optimized conditions (see, Example 3 and FIG. 4), the assay was also evaluated for dilution linearity; specificity and selectivity; impact of hemolyzed and lipemic samples; stability and ability to monitor the assay.

### A. Dilution Linearity

Assays were performed as described above with serial dilutions of spiked sample of IDUA and IDS at 1000 ng/mL and 30.7 μg/mL in neat heat inactivated plasma, respectively.

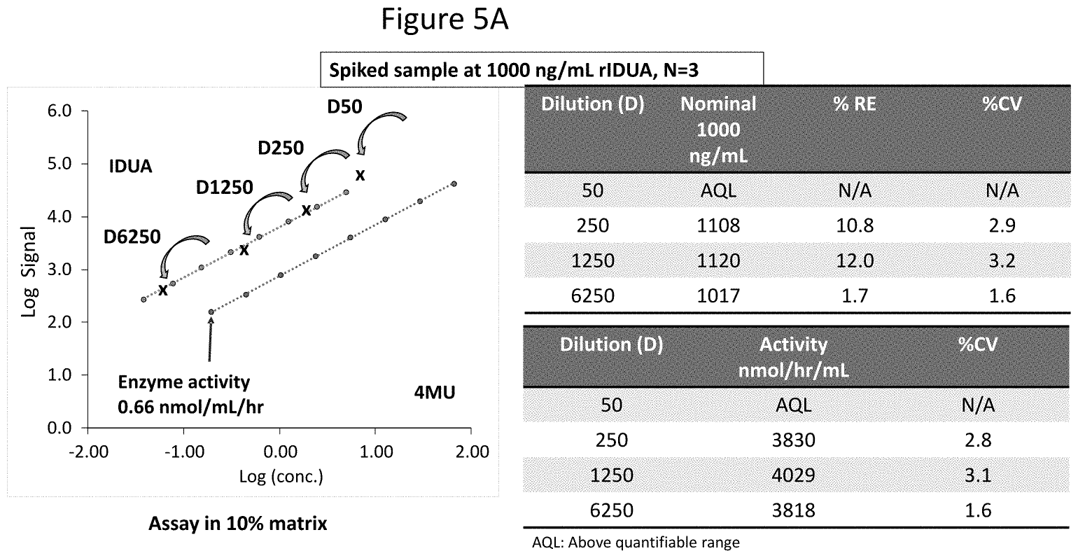

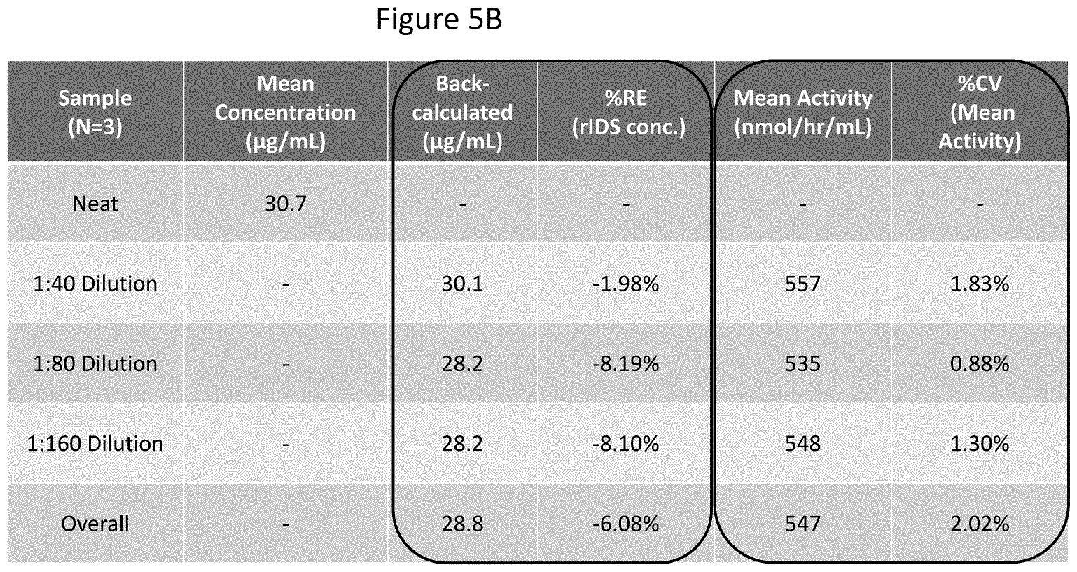

As shown in FIG. 5B, the IDS assay demonstrated dilution linearity. As described in Example 6, the IDUA assay also demonstrated dilution linearity.

### B. Specificity and Selectivity

IDS assays were performed above to evaluate specificity and selectivity when using rIDS as a reference standard. In brief, 10 heat inactivated individual healthy donors were spiked with rIDS at 0.1 μg/mL. Both spiked and unspiked samples were measured at MRD of 1:10.

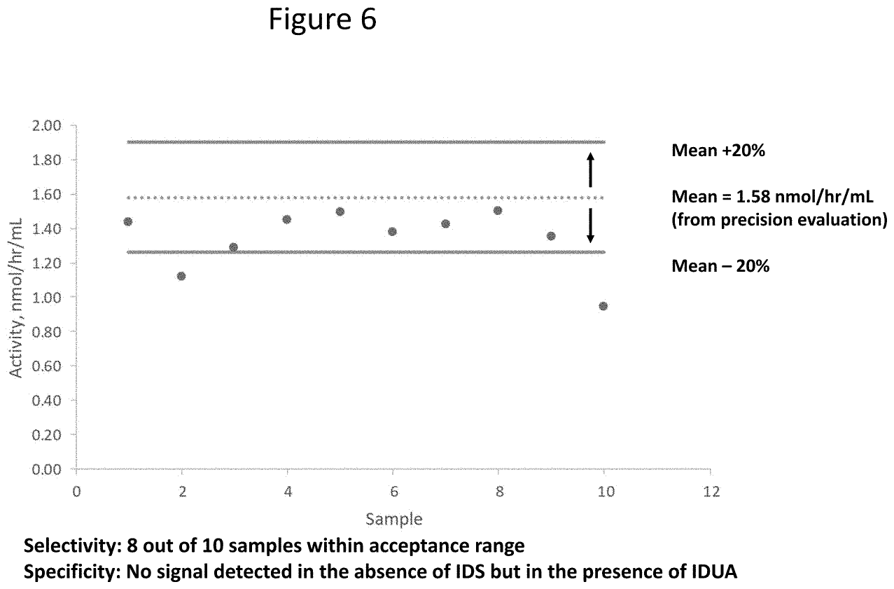

As shown in FIG. 6, the assays exhibited both selectivity (8 of 10 samples within the acceptance range) and specificity (no signal detected in the absence of IDS but in the presence of IDUA).

### C. Hemolyzed and Lipemic Samples (IDUA)

Assays were performed using the IDUA assay using either hemolyzed (H) or lipemic (L) samples from two different donors at varying dilutions and the activity was measured.

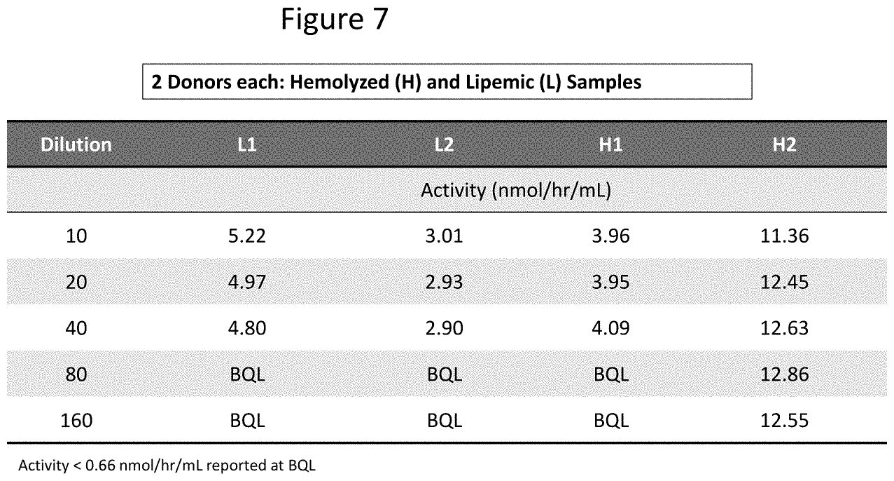

As shown in FIG. 7, different dilutions for a given sample gave similar activity within the assay range and no interference was observed.

### D. Stability (Freeze/Thaw)

IDS and IDUA assays were performed as described above except samples were subject to freeze/thaw cycles 1, 2, 3, 4 or 5 times.

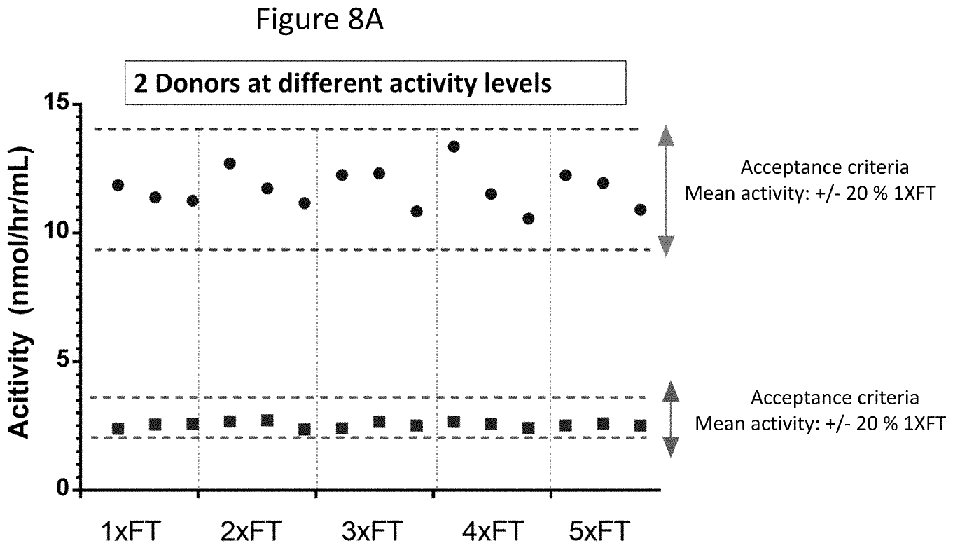

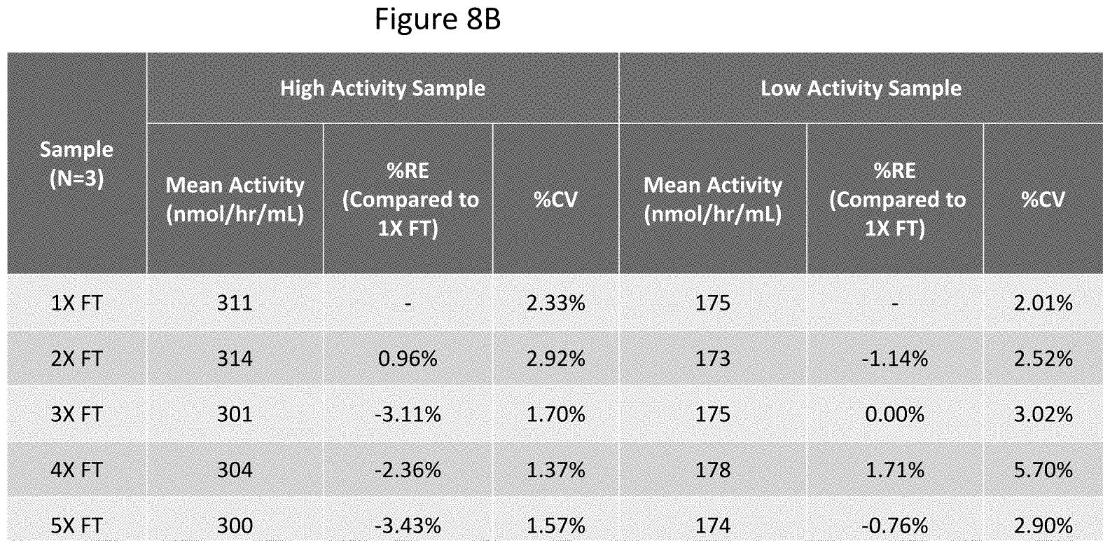

As shown in FIGS. 8A and 8B (IDS) and Example 6 (IDUA), all samples were stable (retained activity levels) for 5 freeze and thaw cycles.

### E. Donor Range

Assays were performed on samples obtained from healthy donors and to assess IDS activity in healthy donors.

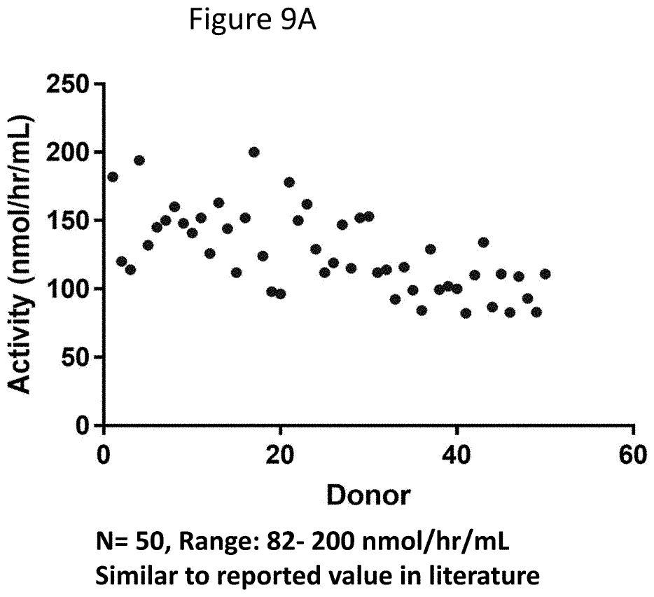

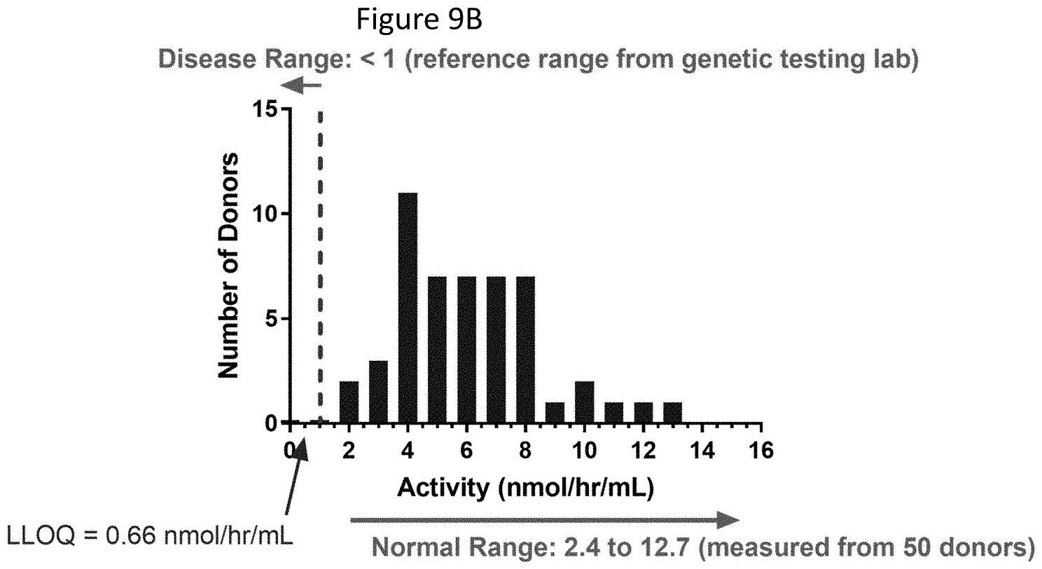

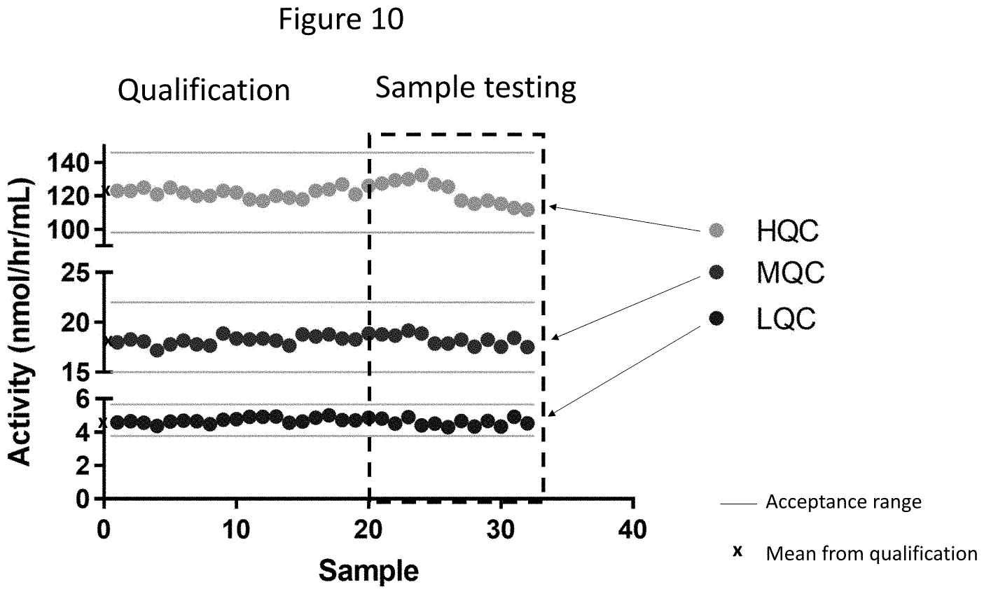

As shown in FIGS. 9A and 9B, the assays described herein provide results for healthy donors in keeping with those reported in the literature, confirming the assays function as intended. In addition, as shown in FIG. 10, results from HQC, MQC and LQC all fell within the acceptance range. Similar results for IDUA are described in Example 6.

### F. Implementation

These data demonstrate that including recombinant rIDS as a reference standard in the first reaction of the IDS assay and recombinant rIDUA as a reference standard in the single-step IDUA assay provided improved quantification and reproducibility. Specifically, for assessing in vivo therapies, 2 standard curves (recombinant IDS, 4MU) and two sets of quality controls (3 levels) for assay monitoring are used in each. Data is determined acceptable, the mean back calculated concentrations for at least 75% of the standards must have RE within ±20% except at ULOQ and LLOQ with RE within ±25%. Calibration standards should have TE≤30% except for LLOQ at ≤40%. Calibration standards except for LLOQ can be masked; however, a minimum of 6 passing calibration points must be present including LLOQ. The % CV of the blank-corrected relative fluorescence units (RFU) for each standard must be less than or equal to 20%. The calibration curve should have r²>0.98.

Each sample analysis plate will contain two sets of quality controls (HQC, MQC, and LQC of rIDS or rIDUA spiked into heat inactivated normal human plasma), run in duplicate. The mean concentration for each set of controls will be back calculated from the IDS standard curve. The mean activity for each set of controls will be back calculated from the 4MU standard curve. For data to be accepted, at least 4 out of the 6 (67%) controls must have % nominal values equal to ±20% of the nominal enzyme (IDS or IDUA) concentration and the corresponding QC enzyme activity within the established activity range from method qualification for each control, as shown below:

QC IDS Enzyme Activity Range

| | Mean Activity from | Acceptable Activity Range |
|---|---|---|
| | BAL-17-080-085.02-REP | (Mean Activity ± 20%) |
| QC | nmol/mL/hr | nmol/mL/hr |
| HQC | 122 | 98-146 |
| MQC | 18.2 | 14.6-21.9 |
| LQC | 4.71 | 3.77-5.66 |

QC IDUA Enzyme Activity Range

| | Mean Activity from | Acceptable Activity Range |
|---|---|---|
| | BAL-17-080-083-REP | (Mean Activity ± 20%) |
| QC | nmol/mL/hr | nmol/mL/hr |
| HQC | 143 | 114-171 |
| MQC | 21.8 | 17.4-26.2 |
| LQC | 3.37 | 2.70-4.04 |

No more than one control from each level can fail the acceptance. Acceptable calculated values must also have % CVs of blank-corrected RFU equal to or less than 20%. Finally, the controls at each level must meet these criteria for acceptance.

### Example 5

The assay in Example 2 was used to assess plasma IDS in MPS II subjects (receiving or not receiving ERT) treated with gene therapy reagents (nuclease-mediated integration using AAV ZFN and an IDS transgenes) as described in U.S. Provisional Application No. 62/802,558. In particular, plasma IDS activity was measured at trough, which was defined as in the period immediately prior to ERT dosing when possible, and no less than 96 hours after the subject's last ERT infusion

Samples obtained less than 96 hours post-ERT dosing were excluded. In this assay, MPS II baseline subjects are <10 nmol/mL/hr IDS activity, with a baseline in a healthy population being >82 nmol/mL/hr. A substantial increase in plasma IDS activity was observed in one subject at a high dose of the ZFN/IDS reagents, however this decreased after the development of mild transaminitis. In all, plasma activity levels from the first six patients enrolled across all three cohorts of the study, at 24 weeks post-treatment were compared to baseline. Enzyme assay analysis detected small increases in IDS activity in the plasma of the two subjects in at the mid-dose, and in one subject at the high dose. Furthermore, a significant increase in plasma IDS activity was measured in the second patient treated at the high dose, with plasma IDS levels rising to approximately 50 nmol/mL/hr by day 50 post-SB-913 treatment, which is approximately 60% of the lower limit of healthy plasma IDS activity.

Thus, the assays described herein quantitatively measure IDS activity in in vivo gene therapy patients (including those receiving ERT).

In addition, IDS assays are preformed on leukocyte samples essentially as described above for plasma samples, except curves are made in buffer and leukocytes are sonicated. , IDS assays performed on leukocyte samples may also be performed as described below for IDUA. Briefly leukocytes are prepared from whole blood collected and sonicated once or more times (e.g., twice for a total of 30 seconds). Leukocyte lysates are typically diluted at 1:1 ratio (MRD 2) with DPBS/0.2% BSA containing protease inhibitor (Sample Diluent) as described below for IDUA assays. The sample is then mixed at a 1:1 ratio with fluorescent substrate to generate standard curves as described herein.

The IDUA assay contains two calibration curves prepared in sample diluent, an enzyme curve and a 4MU curve. The enzyme curve is used to measure the enzyme concentration and the 4MU curve is used to calculate enzyme activity in leukocytes, including in MPS I patients receiving ERT and/or gene therapy. During validation, 5 levels of quality control samples (ULOQ QC, HQC, MQC, LQC, and LLOQ QC) are included to define the quantifiable range of the assay. QCs can be prepared lysate from healthy donors (endogenous IDUA) or a combination of endogenous sample and recombinant hIDUA spiked into sample diluent. Three levels of quality control samples (HQC, MQC, LQC) are included in each run during sample testing with a minimum of one of the three QC samples being leukocyte lysate prepared from healthy donors. The same assay acceptance as used in plasma assay detailed above is used for leukocyte assay.

### Example 6: IDUA Assays

### A. Plasma

The purpose of this study was to qualify an enzymatic assay for the measurement of alpha-iduronidase (IDUA) enzyme activity in plasma that utilizes a 4-methyl-lubelliferone conjugated substrate and fluorometry. The assay contained two calibration curves, an enzyme curve and a 4MU curve, and was performed at a minimum required dilution (MRD) of 1:10. The enzyme curve was used to measure the enzyme concentration and the 4MU curve was used to calculate enzyme activity, including in MPS I patients receiving ERT and/or gene therapy as described in 62/802,568.

This assay was designed to quantitate the enzyme activity of IDUA in K2EDTA-treated human plasma using rhIDUA to control the assay performance. IDUA is a lysosomal enzyme that catalyzes the hydrolysis of unsulfated alpha-L-iduronosidic linkages in heparan sulfate and dermatan sulfate. This assay uses an artificial 4MU substrate that contains a terminal iduronic acid. The removal of iduronic acid is catalyzed by the IDUA enzyme, thus “releasing” the 4MU fluorescence. However, if no endogenous IDUA enzyme is present within the patient sample, the iduronic acid moiety is prevented from being removed, thereby quenching the fluorescence of the 4MU substrate. Therefore, 4MU fluorescence is positively correlated with IDUA concentration and activity. The upper and lower limits of quantification for IDUA concentration and enzyme activity in this assay are shown below:

LLOQ and ULOQ Values for IDUA Concentration
and Enzyme Activity

| Test Name (for concentration) | IDUA |
|---|---|
| Lower limit of quantitation (LLOQ): | 0.039 ng/mL |
| In-well concentration. Multiply by | |
| 10 for dilution corrected | |
| concentration. | |
| Upper limit of quantitation (ULOQ): | 5.0 ng/mL |
| in-well concentration. Multiply by | |
| 10 for dilution corrected | |
| concentration. | |
| | 4 MU (μM); Enzyme |
| Test Name (for enzyme activity) | activity nmol/mL/hr |
| Lower limit of quantitation (LLOQ): | 0.197 μM (Enzyme Activity: |
| Corrected for MRD (10) and reaction time | 0.66 nmol/mL/hr) |
| (hr) for enzyme activity | |
| Upper limit of quantitation (ULOQ): | 67.1 μM (Enzyme Activity: |
| Corrected for MRD (10) and reaction time | 223.67 mnol/mL/hr) |
| (hr) for enzyme activity | |

All concentration data presented in the report are in-well concentration (at MRD of 1:10). However, enzyme activity is reported following correction for MRD, thus reporting activity in neat plasma at nmol/hr/mL.

Frozen plasma samples were removed from freezer and thawed quickly at 37° C. water bath prior to dilution. Plasma samples were diluted 1:10 with assay diluent (10 μL plasma +90 μL assay diluent) in a separate microcentrifuge tube, wherein the assay diluent was 1×PBS containing 0.2% BSA. In each patient/control tube, 20 diluted plasma +20 μL 0.36 mM substrate (4MU-IDUA) were combined in a microplate and incubated in a 37° C. incubator for 3 hours. 160 μL stop solution was added to each well. 100 μL of each reaction was transferred to a flat white opaque plate. Fluorescence signal was acquired using (365 nm excitation, 450 nm emission) plate reader. Total enzyme activity was determined using the following calculations:

Plasma: Average corrected reading×dilution factor (10)=nmoles of substrate hydrolyzed per 3 hours per mL plasma. Normal plasma values were from 2.44-12.7 nmol/mL/hr (determined from 50 donors). The lower limit of quantification (LLOQ) of enzyme activity was 0.66 nmol/mL/hr. The upper limit of the analytical measurement range for enzyme activity was 223.67 nmol/mL/hr.

### Preparation of the Calibration Standards

### IDUA Standard

Recombinant human IDUA was purchased from R&D Systems. IDUA was provided at 288 μg/mL in a buffer containing 40 mM Sodium Acetate, 400 mM NaCl and 20% (v/v) Glycerol, pH 5.0. The IDUA solution was aliquoted into single use tubes so that a fresh standard curve can be prepared for each assay. IDUA curve was prepared fresh on the day of use using assay diluent containing 10% heat inactivated human plasma.

4MU standard

4-Methylumbelliferone was purchased from Sigma-Aldrich. 4MU was provided as a freeze-dried powder. 4MU was reconstituted in DMSO at 200 mM and aliquoted into single use vials. A fresh 4MU standard curve was prepared for each assay. 4MU curve was prepared fresh on the day of use using assay diluent containing 10% heat inactivated human plasma.

### Preparation of the Quality Control Samples

Batches of quality controls were prepared by spiking recombinant human IDUA into heat- inactivated pooled human plasma at three levels (Low QC, Mid QC and High QC). The aliquots were stored at −65° C. to −85° C. Each assay contained at least 2 separate QCs at each level, run in replicate.

Batches of upper and lower limit of quantification controls, ULOQ and LLOQ, were prepared by spiking recombinant human IDUA into heat-inactivated pooled human plasma. The aliquots were stored at −65° C. to −85° C. These controls were used only in accuracy and precision analysis.

### Data and Statistical Methods

Assay plates were read on a Synergy 2 plate reader using Gen5 software. All values were blank- subtracted (matrix containing control) in Gen5. Blank-corrected RLU data was then imported from Gen5 into Watson™ LIMS v7.4.2 software for all other analysis.

### Summary of Qualification Results

### Summary of Runs

Qualification of the method included assessment of accuracy, intra- and inter-assay precision, selectivity, dilution linearity, donor normal range, short-term stability (freeze-thaw (F/T), and long-term stability (I-month, 3-month, and 6-month). Long-term stability data will be added to this report as an addendum once those assays are performed. All qualification assay runs are listed below:

Summary of runs

| Run | | | |
|---|---|---|---|
| Number | Run Description | Analyst | Result |
| 1 | Enzyme Activity Normal | 1 | Pass |
| | Donors 1-25 Run 1 | | |
| 2 | Enzyme Activity Normal | 1 | Pass |
| | Donors 1-25 Run 2 | | |
| 3 | Intra Assay (Accuracy | 1 | Pass |
| | and Precision) Run 1 | | |
| 4, 5 | Intra Assay (Accuracy | 1, 2 | Pass |
| | and Precision) Runs 2 & 3 | | |
| 6 | Dilution Linearity | 1 | Fail ¹ |
| 7 | Matrix Interference | 1 | Pass |
| 8 | Intra Assay (Accuracy | 1 | Pass |
| | and Precision) Run 4 | | |
| 9 | Freeze/thaw stability - | 1 | Pass |
| | high activity sample | | |
| 10 | Freeze/thaw stability- | 1 | Pass |
| | low activity sample | | |
| 11 | Hemolytic and Lipemic | 1 | Pass |
| | selectivity | | |
| 12 | Dilution Linearity | 1 | Fail² |
| 13 | Dilution Linearity | 1 | Pass |

¹ Blank matrix controlsfor dilution linearity samples not included on plate.
²Did not perform 3 dilutions of the dilution linearity samples within quantitative range of assay.

### Calibration Curve Performance and Sensitivity Results

An 8-point titration curve of rhIDUA and an 8-point 4MU product curve were evaluated for use as standard curves for the assessment of human IDUA enzyme activity in pooled human plasma. Minimum required dilution of the assay is at 10. The concentrations were chosen based on method development. The in-well concentrations evaluated for rhIDUA during qualification were 5, 2.5, 1.25, 0.625, 0.313, 0.156, 0.078, and 0.039 ng/mL. The reproducibility of the rhIDUA standard curve, as determined by the % Bias and CVs of the individual activity standard dilutions, had an accurate and precise detection range of 5.0 ng/mL to 0.039 ng/mL as shown below:

rIDUA Calibration Curve

| | Run | | | | | | | |
|---|---|---|---|---|---|---|---|---|
| | 5 | 2.5 | 1.25 | 0.625 | 0.313 | 0.156 | 0.078 | 0.039 |
| | (ng/mL) | (ng/mL) | (ng/mL) | (ng/mL) | (ng/mL) | (ng/mL) | (ng/mL) | (ng/mL) |
| IND 1-25 | 4.809 | 2.502 | 1.283 | 0.644 | 0.316 | 0.156 | 0.077 | 0.039 |
| IND 26-50 | 4.796 | 2.520 | 1.280 | 0.639 | 0.316 | 0.157 | 0.077 | 0.038 |
| Selectivity (matrix | 4.809 | 2.512 | 1.275 | 0.640 | 0.317 | 0.157 | 0.077 | 0.038 |
| int) | | | | | | | | |
| Selectivity | 4.796 | 2.501 | 1.272 | 0.645 | 0.318 | 0.158 | 0.078 | 0.038 |
| (hemolysis and | | | | | | | | |
| lipemia) | | | | | | | | |
| Dilution Linearity | 4.717 | 2.532 | 1.278 | 0.647 | 0.320 | 0.157 | 0.077 | 0.038 |
| High activity FIT | 4.985 | 2.547 | 1.273 | 0.628 | 0.306 | 0.148 | 0.076 | 0.042 |
| stability | | | | | | | | |
| Low activity F/T | 4.849 | 2.514 | 1.272 | 0.639 | 0.314 | 0.156 | 0.077 | 0.039 |
| stability | | | | | | | | |
| ACC and PRE 1 | 4.782 | 2.493 | 1.278 | 0.646 | 0.319 | 0.158 | 0.078 | 0.038 |
| ACC and PRE 2 | 4.910 | 2.546 | 1.280 | 0.630 | 0.309 | 0.152 | 0.075 | 0.041 |
| ACC and PRE 3 | 4.774 | 2.515 | 1.276 | 0.641 | 0.321 | 0.158 | 0.077 | 0.038 |
| ACC and PRE 4 | 4.831 | 2.500 | 1.269 | 0.639 | 0.319 | 0.158 | 0.078 | 0.038 |
| n | 11 | 11 | 11 | 11 | 11 | 11 | 11 | 11 |
| Overall Mean | 4.824 | 2.517 | 1.276 | 0.640 | 0.316 | 0.156 | 0.077 | 0.039 |
| S.D. | 0.072 | 0.018 | 0.004 | 0.006 | 0.005 | 0.003 | 0.001 | 0.001 |
| % CV | 1.49 | 0.73 | 0.33 | 0.95 | 1.47 | 2.08 | 1.19 | 3.57 |
| % Bias | −3.53 | 0.66 | 2.08 | 2.36 | 0.89 | −0.01 | −1.39 | −0.98 |
| % TE | 5.02 | 1.39 | 2.41 | 3.31 | 2.36 | 2.09 | 2.58 | 4.55 |

rIDUA Curve Fit Parameters

| Run | Slope | y-intercept | R-Squared |
|---|---|---|---|
| IND 1-25 | 0.9506 | 4.0047 | 0.9998 |
| IND 26-50 | 0.9484 | 4.0075 | 0.9998 |
| Selectivity (matrix int) | 0.9537 | 3.9117 | 0.9998 |
| Selectivity (hemolysis and | 0.9446 | 3.9251 | 0.9998 |
| lipemia) | | | |
| Dilution Linearity | 0.9426 | 3.9137 | 0.9996 |
| High activity F/T stability | 0.9302 | 3.9131 | 0.9995 |
| Low activity F/T stability | 0.9537 | 3.9024 | 0.9999 |
| ACC and PRE 1 | 0.9618 | 3.8038 | 0.9997 |
| ACC and PRE 2 | 0.9243 | 3.8703 | 0.9997 |
| ACC and PRE 3 | 0.9449 | 3.8754 | 0.9998 |
| ACC and PRE 4 | 0.9564 | 3.8358 | 0.9998 |

The reproducibility of the 4MU product curve, as determined by the % Bias ((measured-nominal)/nominal*100) and CVs of the individual activity standard dilutions, had an accurate and precise detection range of 0.197 μM to 67.1 μM. Both curves met the acceptance as outlined in the qualification protocol. The 4MU calibration curves for the runs assessing normal individual plasma (IND 1-25 and IND 26-50) were analyzed separately. The 4MU curves for those runs, although having the same LLOQ as all other runs, were prepared via alternate dilution, ranging from 35.5 μM to 0.197 μM. These data are presented below:

4MU Calibration Curve (passing Runs 3-13)

| | Activity (nmol/mL/hr) | | | | | | | |
|---|---|---|---|---|---|---|---|---|
| | 223.67 | 97.33 | 42.33 | 18.40 | 8.00 | 3.47 | 1.51 | 0.66 |
| | Run | | | | | | | |
| | 67.1 | 29.2 | 12.7 | 5.52 | 2.4 | 1.04 | 0.453 | 0.197 |
| | (μM) | (μM) | (μM) | (μM) | (μM) | (μM) | (μM) | (μM) |
| Selectivity (matrix int) | 63.472 | 29.620 | 13.071 | 5.678 | 2.435 | 1.044 | 0.451 | 0.19 |
| Selectivity (hemolysis and | 62.880 | 29.486 | 13.119 | 5.721 | 2.451 | 1.062 | 0.447 | 0.19 |
| lipemia) | | | | | | | | |
| Dilution Linearity | 62.259 | 29.670 | 13.182 | 5.739 | 2.465 | 1.052 | 0.446 | 0.19 |
| High activity F/T stability | 65.749 | 29.822 | 12.926 | 5.523 | 2.340 | 1.035 | 0.458 | * |
| Low activity F/T stability | 64.049 | 29.490 | 12.999 | 5.655 | 2.434 | 1.053 | 0.446 | 0.19 |
| ACC and PRE I | 64.498 | 29.398 | 13.042 | 5.677 | 2.424 | 1.043 | 0.433 | 0.20 |
| ACC and PRE 2 | 64.870 | 29.192 | 13.006 | 5.598 | 2.427 | 1.053 | 0.455 | 0.19 |
| ACC and PRE 3 | 63.971 | 29.629 | 13.066 | 5.665 | 2.431 | 1.040 | 0.436 | 0.20 |
| ACC and PRE 4 | 64.051 | 29.386 | 13.032 | 5.657 | 2.423 | 1.047 | 0.461 | 0.19 |
| | 9 | 9 | 9 | 9 | 9 | 9 | 9 | 8 |
| Overall Mean | 63.98 | 29.52 | 13.05 | 5.66 | 2.43 | 1.05 | 0.45 | 0.19 |
| S.D. | 1.039 | 0.187 | 0.074 | 0.064 | 0.035 | 0.008 | 0.010 | 0.004 |
| % CV | 1.62 | 0.63 | 0.56 | 1.14 | 1.44 | 0.78 | 2.13 | 2.09 |
| % Bias | −4.65 | 1.10 | 2.75 | 2.49 | 1.07 | 0.73 | −1.06 | −2.39 |
| % TE | 6.28 | 1.73 | 3.31 | 3.62 | 2.51 | 1.50 | 3.18 | 4.48 |

4MU Calibration Curve (Runs 1-2)

| | Activity (nmol/mL/hr) | | | | | | | |
|---|---|---|---|---|---|---|---|---|
| | 118.33 | 5.57 | 26.83 | 12.77 | 6.20 | 2.90 | 1.38 | 0.66 |
| | Run | | | | | | | |
| | 35.5 | 16.9 | 8.05 | 3.83 | 1.86 | 0.869 | 0.414 | 0.197 |
| | (μM) | (μM) | (μM) | (μM) | (μM) | (μM) | (μM) | (μM) |
| Selectivity (matrix int) | 34.597 | 17.011 | 8.154 | 3.907 | 1.854 | 0.875 | 0.411 | 0.195 |
| Selectivity (hemolysis and | 34.552 | 16.952 | 8.156 | 3.930 | 1.849 | 0.877 | 0.415 | 0.193 |
| lipemia) | | | | | | | | |
| n | 2 | 2 | 2 | 2 | 2 | 2 | 2 | 2 |
| Overall Mean | 34.57 | 16.98 | 8.16 | 3.92 | 1.85 | 0.88 | 0.41 | 0.19 |
| S.D. | 0.031 | 0.042 | 0.001 | 0.016 | 0.004 | 0.001 | 0.003 | 0.001 |
| % CV | 0.09 | 0.25 | 0.01 | 0.42 | 0.19 | 0.15 | 0.65 | 0.71 |
| % Bias | −2.61 | 0.48 | 1.30 | 2.31 | −0.45 | 0.81 | −0.26 | −1.50 |
| % TE | 2.70 | 0.73 | 1.32 | 2.73 | 0.64 | 0.96 | 0.91 | 2.21 |

4MU Curve Fit Parameters

| Run | Slope | y-intercept | R-Squared |
|---|---|---|---|
| IND 1-25 | 0.96339 | 3.0194 | 0.9999 |
| IND 26-50 | 0.9608 | 3.0237 | 0.9999 |
| Selectivity (matrix int) | 0.9539 | 2.9731 | 0.9998 |
| Selectivity (hemolysis and | 0.9547 | 2.9449 | 0.9997 |
| lipemia) | | | |
| Dilution Linearity | 0.9669 | 2.9159 | 0.9996 |
| High activity F/T stability | 0.9524 | 2.9409 | 0.9999 |
| Low activity F/T stability | 0.9728 | 2.9136 | 0.9998 |
| ACC and PRE I | 0.9674 | 2.8711 | 0.9998 |
| ACC and PRE 2 | 0.9280 | 2.9267 | 0.9999 |
| ACC and PRE 3 | 0.9579 | 2.8824 | 0.9998 |
| ACC and PRE 4 | 0.9703 | 2.8601 | 0.9998 |

Accuracy and Precision of rhIDUA Controls

Evaluation of in-well upper limit of quantification (5.0 ng/mL), high (4.0 ng/mL), mid (0.6 ng/mL) low (0.1 ng/mL) and lower limit of quantification (0.039 ng/mL) of rhIDUA concentration controls (ULOQ, HQC, MQC, LQC, and LLOQ, respectively) was performed by interpolating concentrations of the rhIDUA controls from the rhIDUA standard curve and compared to the nominal concentration.

The performances of the controls are presented with the measured concentration and the accuracy (%Theoretical and % Bias). %Theoretical and %Bias are calculated using the formulas:

% Theoretical=(Measured Concentration/Nominal Concentration)×100%

Bias (%Relative Error)=[(Measured Concentration/Nominal Concentration)/Nominal Concentration]×100

Precision (intra and inter-assay precision) is represented by the coefficient of variation (CV) expressed as a percentage calculated using single factor ANOVA analysis as shown below:

Accuracy and Precision of rIDUA Controls

| | | LLOQ | LQC | MQC | HQC | ULOQ |
|---|---|---|---|---|---|---|
| | Run | (0.039 ng/mL) | (0.1 ng/mL) | (0.6 ng/mL) | (4 ng/mL) | (5 ng/mL) |
| | 3 (ACC and | 0.042 | 0.105 | 0.644 | 4.44 | 5.48 |
| | PRE run 1) | 0.039 | 0.105 | 0.674 | 4.41 | 5.26 |
| | | 0.038 | 0.103 | 0.698 | 4.44 | 5.42 |
| | | 0.038 | 0.107 | 0.688 | 4.27 | 5.39 |
| | | 0.037 | 0.099 | 0.675 | 4.39 | 5.38 |
| | 4 (ACC and | 0.034 | 0.085 | 0.569 | 4.26 | 5.05 |
| | PRE run 2) | 0.033 | 0.089 | 0.608 | 4.19 | 5.01 |
| | | 0.033 | 0.088 | 0.614 | 4.21 | 5.03 |
| | | 0.032 | 0.088 | 0.619 | 4.36 | 5.05 |
| | | 0.031 | 0.088 | 0.590 | 4.15 | 5.08 |
| | 5 (ACC and | 0.034 | 0.092 | 0.612 | 3.99 | 5.09 |
| | PRE run 3) | 0.037 | 0.092 | 0.613 | 4.09 | 5.07 |
| | | 0.034 | 0.092 | 0.618 | 4.12 | 4.90 |
| | | 0.034 | 0.092 | 0.614 | 4.15 | 5.07 |
| | | 0.034 | 0.089 | 0.591 | 4.09 | 4.96 |
| | 8 (ACC and | 0.041 | 0.108 | 0.686 | 4.39 | 5.52 |
| | PRE run 4) | 0.041 | 0.107 | 0.680 | 4.47 | 5.45 |
| | | 0.040 | 0.105 | 0.688 | 4.45 | 5.53 |
| | | 0.041 | 0.106 | 0.702 | 4.45 | 5.55 |
| | | 0.040 | 0.115 | 0.700 | 4.50 | 5.42 |
| Mean | | 0.037 | 0.098 | 0.644 | 4.29 | 5.24 |
| S.D. | | 0.00 | 0.01 | 0.04 | 0.16 | 0.22 |
| % CV | | 9.59 | 9.34 | 6.73 | 3.68 | 4.23 |
| % Theoretical | | 94.0 | 97.8 | 107.3 | 107.3 | 104.7 |
| % Bias | | −6.02 | −2.24 | 7.35 | 7.28 | 4.72 |
| n | | 20 | 20 | 20 | 20 | 20 |

Precision (ANOVA Analysis) of rlDUA Controls

| | LLOQ | LQC | MQC | HQC | ULOQ |
|---|---|---|---|---|---|
| | (0.039 | (0.1 | (0.6 | (4 | (5 |
| Nominal Conc. | ng/mL) | ng/mL) | ng/mL) | ng/mL) | ng/mL) |
| Mean Observed Conc. | 0.037 | 0.098 | 0.644 | 4.29 | 5.24 |
| % Bias | −8.4 | −2.2 | 7.3 | 7.3 | 4.7 |
| Between Run Precision | 10.0 | 10.0 | 7.0 | 3.8 | 4.5 |
| (% CV) | | | | | |
| Within Run Precision | 3.7 | 2.8 | 2.5 | 1.5 | 1.3 |
| (% CV) | | | | | |
| Total Variation (% CV) | 10.6 | 10.4 | 7.5 | 4.1 | 4.7 |
| n | 20 | 20 | 20 | 20 | 20 |
| Number of Runs | 4 | 4 | 4 | 4 | 4 |

Thus, accuracy and precision analysis met the acceptance as outlined in the qualification protocol.

Precision of IDUA Activity and Determination of Assay Acceptance Criteria

Evaluation of the activity of ULOQ, HQC, MQC, LQC, and LLOQ was performed by interpolating IDUA activity using the 4MU standard curve. The activity data is presented as nmol/mL/hr. This analysis allowed the determination of acceptable activity ranges of the QCs for plate acceptance criteria. Plates are accepted if the QCs fall within ±20% of the calculated means shown below:

Accuracy and Precision of rIDUA Controls

| | | LLOQ | LQC | MQC | HQC | ULOQ |
|---|---|---|---|---|---|---|
| | Run | (0.039 ng/mL) | (0.1 ng/mL) | (0.6 ng/mL) | (4 ng/mL) | (5 ng/mL) |
| | 3 (ACC and | 1.31 | 3.26 | 19.8 | 135 | 166 |
| | PRE run 1) | 1.21 | 3.25 | 20.7 | 134 | 160 |
| | | 1.20 | 3.21 | 21.5 | 135 | 165 |
| | | 1.18 | 3.33 | 21.1 | 130 | 164 |
| | | 1.16 | 3.08 | 20.7 | 134 | 163 |
| | 4 (ACC and | 1.18 | 2.96 | 19.7 | 147 | 174 |
| | PRE run 2) | 1.17 | 3.13 | 21.1 | 144 | 172 |
| | | 1.15 | 3.07 | 21.3 | 145 | 173 |
| | | 1.12 | 3.08 | 21.5 | 150 | 174 |
| | | 1.09 | 3.06 | 20.5 | 143 | 175 |
| | 5 (ACC and | 1.30 | 3.45 | 22.3 | 142 | 180 |
| | PRE run 3) | 1.42 | 3.43 | 22.4 | 145 | 180 |
| | | 1.29 | 3.45 | 22.5 | 146 | 174 |
| | | 1.30 | 3.44 | 22.4 | 148 | 180 |
| | | 1.27 | 3.32 | 21.6 | 145 | 176 |
| | 8 (ACC and | 1.45 | 3.77 | 23.2 | 145 | 182 |
| | PRE run 4) | 1.45 | 3.73 | 23.0 | 148 | 179 |
| | | 1.42 | 3.65 | 23.3 | 147 | 182 |
| | | 1.44 | 3.70 | 23.8 | 147 | 183 |
| | | 1.41 | 3.99 | 23.7 | 149 | 178 |
| Mean | | 1.28 | 3.37 | 21.8 | 143 | 174 |
| S.D. | | 0.12 | 0.28 | 1.23 | 5.91 | 6.99 |
| % CV | | 9.45 | 8.40 | 5.66 | 4.14 | 4.02 |
| n | | 20 | 20 | 20 | 20 | 20 |
| Assay | +20% | 1.53 | 4.04 | 26.2 | 171 | 209 |
| acceptance | −20% | 1.02 | 2.70 | 17.4 | 114 | 139 |
| criteria for | | | | | | |
| assays (HQC, | | | | | | |
| MQC, LQC | | | | | | |
| will be used | | | | | | |
| for assay | | | | | | |
| acceptance) | | | | | | |
| (nmol/mL/hr) | | | | | | |

### Dilution Linearity

Neat, heat-inactivated human plasma from 3 individuals (DL1, DL2, and DL3) was spiked with rhIDUA at a concentration of 1000 ng/mL. After the assay MRD of 1 : 1 0 , the rhIDUA concentration of the samples (100) was still well above the ULOQ of the assay. Therefore, to investigate prozone effect and determine dilution linearity, all samples were assayed at 4 dilution factors, 50 (A), 250 (B), and 1250 (C), and 6250 (D) while maintaining MRD. The back calculated values were then compared to the original sample concentration of 1000 ng/mL to confirm acceptable dilution linearity as shown below.

Accuracy of dilution linearity IDUA concentration

| | Conc. in neat | | | | |
|---|---|---|---|---|---|
| Sample | HI plasma | Dilution | Mean Conc. | back-cal | |
| Name | (ng/mL) | factor | (ng/mL) | (ng/mL) | % Bias |
| DL1A | 1000 | 50 | ALQ ¹ | N/A | N/A |
| DL2A | 1000 | 50 | ALQ ¹ | N/A | N/A |
| DL3A | 1000 | 50 | ALQ ¹ | N/A | N/A |
| DL1 B | 1000 | 250 | 4.31 | 1076.4 | 7.64 |
| DL2B | 1000 | 250 | 4.56 | 1140.2 | 14.02 |
| DL3B | 1000 | 250 | 4.43 | 1106.8 | 10.68 |
| DL1 C | 1000 | 1250 | 0.87 | 1084.6 | 8.46 |
| DL2C | 1000 | 1250 | 0.93 | 1156.3 | 15.63 |
| DL3C | 1000 | 1250 | 0.89 | 1117.7 | 11.77 |
| DL1 D | 1000 | 6250 | 0.16 | 998.1 | −0.19 |
| DL2D | 1000 | 6250 | 0.16 | 1029.4 | 2.94 |
| DL3D | 1000 | 6250 | 0.16 | 1022.1 | 2.21 |

¹ Above quantitative limit

All samples diluted 50 times were still above ULOQ and registered as such. All quantifiable samples were within the acceptable range of ±20% Bias.

Furthermore, The IDUA activity of all dilution linearity samples was determined, while correcting for dilution factor. Regardless of dilution factor, the precision of all samples was within ±20% CV as shown below. The dilution factors in were corrected for incubation time of the assay (3 hours). Enzyme activity is reported as nmol/hr/mL, therefore the dilution factor is divided by 3.

Precision of Dilution Linearity IDUA activity

| Sample | Dilution | Mean Activity | |
|---|---|---|---|
| Name | factor | (nmol/mL/hr) | % CV |
| DL1A | 16.67 | AQL1 | |
| DL2A | 16.67 | AQL1 | N/A |
| DL3A | 16.67 | AQL1 | |
| DL1B | 83.33 | 3723.96 | |
| DL2B | 83.33 | 3938.91 | 2.81 |
| DL3B | 83.33 | 3826.33 | |
| DL1C | 416.67 | 3906.19 | |
| DL2C | 416.67 | 4157.71 | 3.12 |
| DL3C | 416.67 | 4022.30 | |
| DL1D | 2083.33 | 3750.43 | |
| DL2D | 2083.33 | 3865.16 | 1.57 |
| DL3D | 2083.33 | 3838.47 | |

| | |
|---|---|
| Overall % CV | 3.47 |

1Above quantitative limit

FIG. 5A also shows results for dilution studies.

### Selectivity

Selectivity runs were conducted to determine if components of the assay matrix (i.e. human plasma) other than the desired target, IDUA, could alter the results. To test this, 10 heat-inactivated individual plasma samples were spiked with rh1DUA at the LLOQ (0.39 ng/mL, in-well at MRD 1:10 at 0.039 ng/mL; INDx HI Spiked). The same individual heat inactivated neat samples (INDx HI) were run simultaneously. As shown below, all null samples gave no detectable response while all spiked samples were within ±20% of the nominal concentration. When rIDUA was added to the heat inactivated plasma, measurable enzyme activity with enzyme concentration within ±20% of the nominal concentration was obtained.

Selectivity by matrix interference - IDUA Concentration

| Sample | Mean Conc. | Nominal Conc. | |
|---|---|---|---|
| Name | (ng/mL) | (ng/mL) | % Bias |
| IND 1 HI | BQL | null | N/A |
| IND2 HI | BQL | null | N/A |
| IND3 HI | BQL | null | N/A |
| IND4 HI | BQL | null | N/A |
| INDS HI | BQL | null | N/A |
| IND6 HI | BQL | null | N/A |
| IND7 HI | BQL | null | N/A |
| IND8 HI | BQL | null | N/A |
| IND9 HI | BQL | null | N/A |
| IND 10 HI | BQL | null | N/A |
| IND 1 HI Spiked | 0.040 | 0.039 | 3.16 |
| IND2 HI Spiked | 0.036 | 0.039 | −6.76 |
| IND3 HI Spiked | 0.036 | 0.039 | −6.62 |
| IND4 HI Spiked | 0.038 | 0.039 | −2.80 |
| IND5 HI Spiked | 0.039 | 0.039 | −0.11 |
| IND6 HI Spiked | 0.038 | 0.039 | −2.94 |
| IND7 HI Spiked | 0.038 | 0.039 | −2.66 |
| IND8 HI Spiked | 0.039 | 0.039 | −0.67 |
| IND9 HI Spiked | 0.040 | 0.039 | 1.60 |
| INDI0 HI Spiked | 0.037 | 0.039 | −5.34 |

BQL = below quantitative limit

Furthermore, the activity for the spiked individuals was within +20% of the mean LLOQ activity determined as detailed above. Results are shown below:

Selectivity by matrix interference - IDUA activity

| Sample | Mean Activity | Mean Activity | |
|---|---|---|---|
| Name | (nmol/mL/hr) | (nmol/mL/hr)¹ | % Bias |
| IND1 HI | BQL | null | N/A |
| IND2 HI | BQL | null | N/A |
| IND3 HI | BQL | null | N/A |
| IND4 HI | BQL | null | N/A |
| IND5 HI | BQL | null | N/A |
| IND6 HI | BQL | null | N/A |
| IND7 HI | BQL | null | N/A |
| IND8 HI | BQL | null | N/A |
| IND9 HI | BQL | null | N/A |
| IND10 HI | BQL | null | N/A |
| IND1 HI Spiked | 1.29 | 1.28 | 0.9 |
| IND2 HI Spiked | 1.17 | 1.28 | −8.8 |
| IND3 HI Spiked | 1.17 | 1.28 | −8.6 |
| IND4 HI Spiked | 1.22 | 1.28 | −4.9 |
| IND5 HI Spiked | 1.25 | 1.28 | −2.2 |
| IND6 HI Spiked | 1.22 | 1.28 | −5.0 |
| IND7 HI Spiked | 1.22 | 1.28 | −4.7 |
| IND8 HI Spiked | 1.24 | 1.28 | −2.8 |
| IND9 HI Spiked | 1.27 | 1.28 | −0.6 |
| IND10 HI Spiked | 1.19 | 1.28 | −7.4 |

BQL = below quantitative limit
¹Calculated from accuracy and precision

Neat hemolytic and lipemic individual samples were tested at multiple dilutions while maintaining MRD to determine if interference was present. The back calculated activity, after correcting for dilution, was compared to the MRD of 1:10 activity value. VL denotes the visibly lipemic samples and VH denotes the visibly hemolytic samples. The results are shown in below.

Selectivity in hemolytic and lipemic samples - IDUA activity

| | | | Mean activity of | |
|---|---|---|---|---|
| Sample | Dilution | Mean Activity | 10x dilution | |
| Name | Factor | (nmol/mL/hr) | (nmol/mL/hr) | % Bias |
| VL1 A | 10 | 5.22 | | N/A |
| VL1 B | 20 | 4.97 | 5.22 | −4.73 |
| VL1 C | 40 | 4.80 | 5.22 | −8.03 |
| VL1 D | 80 | BQL | 5.22 | N/A |
| VL1 E | 160 | BQL | 5.22 | N/A |
| VL2 A | 10 | 3.01 | | N/A |
| VL2 B | 20 | 2.93 | 3.01 | −2.60 |
| VL2 C | 40 | 2.90 | 3.01 | −3.53 |
| VL2 D | 80 | BQL | 3.01 | N/A |
| VL2 E | 160 | BQL | 3.01 | N/A |
| VH1 A | 10 | 3.96 | | N/A |
| VH1 B | 20 | 3.95 | 3.96 | −0.40 |
| VH1 C | 40 | 4.09 | 3.96 | 3.28 |
| VH1 D | 80 | BQL | 3.96 | N/A |
| VH1 E | 160 | BQL | 3.96 | N/A |
| VH2 A | 10 | 11.36 | | N/A |
| VH2 B | 20 | 12.45 | 11.36 | 9.63 |
| VH2 C | 40 | 12.63 | 11.36 | 11.19 |
| VH2 D | 80 | 12.86 | 11.36 | 13.26 |
| VH2 E | 160 | 12.55 | 11.36 | 10.51 |

As shown, all back calculated quantifiable activities were within ±20% of the MRD of 1:10 sample value.

### Freeze/Thaw Stability

Stability assessments were performed on individual healthy donor samples subjected to freeze and thaw conditions. After screening all normal individual plasma samples, a sample with a high activity and a sample with a low activity were chosen to undergo freeze thaw stability testing. Samples underwent 5 freeze-thaw cycles and were tested in triplicate (A, B, and C) on a single plate. Concentrations were interpolated from the rhIDUA curve and activity was determined from the 4MU curve, both shown below. Compared to the 1st freeze-thaw cycle, the % CV for the high and low activity sample of all subsequent freeze-thaw cycles is within the acceptable range for concentration and activity, within 20%.

Freeze/Thaw Stability IDUA Concentration

| | | Mean Conc. | |
|---|---|---|---|
| | | of 1XF/T | |
| Sample | Mean Conc. | triplicate | |
| Name | (ng/mL) | (ng/mL) | % Bias |
| High 1A | 0.33 | | N/A |
| High 1B | 0.32 | 0.32 | N/A |
| High 1C | 0.31 | | N/A |
| High 2A | 0.35 | 0.32 | 10.71 |
| High 2B | 0.33 | 0.32 | 2.04 |
| High 2C | 0.31 | 0.32 | −3.05 |
| High 3A | 0.34 | 0.32 | 6.73 |
| High 3B | 0.34 | 0.32 | 7.34 |
| High 3C | 0.30 | 0.32 | −5.84 |
| High 4A | 0.37 | 0.32 | 16.63 |
| High 4B | 0.32 | 0.32 | 0.21 |
| High 4C | 0.29 | 0.32 | −8.38 |
| High 5A | 0.34 | 0.32 | 6.64 |
| High 5B | 0.33 | 0.32 | 3.98 |
| High 5C | 0.30 | 0.32 | −5.20 |
| Low 1A | 0.066 | 0.069 | N/A |
| Low 1B | 0.070 | | N/A |
| Low 1C | 0.071 | | N/A |
| Low 2A | 0.073 | 0.069 | 6.47 |
| Low 2B | 0.075 | 0.069 | 8.50 |
| Low 2C | 0.065 | 0.069 | −5.84 |
| Low 3A | 0.066 | 0.069 | −3.82 |
| Low 3B | 0.073 | 0.069 | 5.96 |
| Low 3C | 0.069 | 0.069 | 0.22 |
| Low 4A | 0.073 | 0.069 | 6.13 |
| Low 4B | 0.071 | 0.069 | 2.92 |
| Low 4C | 0.066 | 0.069 | −3.74 |
| Low 5A | 0.069 | 0.069 | 0.39 |
| Low 5B | 0.071 | 0.069 | 3.68 |
| Low 5C | 0.069 | 0.069 | 0.14 |

High xA: x can be 1, 2, 3, 4, 5 and represents freeze thaw cycle. A, B, and C mean different aliquot.
Low xA: x can be 1, 2, 3, 4, 5 and represents freeze thaw cycle. A, B, and C mean different aliquot.

Freeze/Thaw Stability IDUA Activity

| Sample | Mean | Mean activity | % Bias |
|---|---|---|---|
| High 1A | 11.9 | 11.5 | N/A |
| High 1B | 11.4 | | N/A |
| High 1C | 11.2 | | N/A |
| High 2A | 12.7 | 11.5 | 10.45 |
| High 2B | 11.7 | 11.5 | 2.00 |
| High 2C | 11.2 | 11.5 | −2.97 |
| High 3A | 12.3 | 11.5 | 6.57 |
| High 3B | 12.3 | 11.5 | 7.16 |
| High 3C | 10.8 | 11.5 | −5.70 |
| High 4A | 13.4 | 11.5 | 16.21 |
| High 4B | 11.5 | 11.5 | 0.20 |
| High 4C | 10.6 | 11.5 | −8.19 |
| High 5A | 12.2 | 11.5 | 6.48 |
| High 5B | 11.9 | 11.5 | 3.88 |
| High 5C | 10.9 | 11.5 | −5.08 |
| Low 1A | 2.39 | 2.51 | N/A |
| Low 1B | 2.55 | | N/A |
| Low 1C | 2.58 | | N/A |
| Low 2A | 2.67 | 2.51 | 6.34 |
| Low 2B | 2.72 | 2.51 | 8.32 |
| Low 2C | 2.36 | 2.51 | −5.72 |
| Low 3A | 2.41 | 2.51 | −3.74 |
| Low 3B | 2.66 | 2.51 | 5.84 |
| Low 3C | 2.51 | 2.51 | 0.22 |
| Low 4A | 2.66 | 2.51 | 6.01 |
| Low 4B | 2.58 | 2.51 | 2.86 |
| Low 4C | 2.42 | 2.51 | −3.66 |
| Low 5A | 2.52 | 2.51 | 0.38 |
| Low 5B | 2.60 | 2.51 | 3.61 |
| Low 5C | 2.51 | 2.51 | 0.14 |

High xA: x can be 1, 2, 3, 4, 5 and represents freeze thaw cycle. A, B, and C mean different aliquot.
Low xA: x can be 1, 2, 3, 4, 5 and represents freeze thaw cycle. A, B, and C mean different aliquot.

Thus, freeze-thawed samples were stable for at least 5 cycles of freeze-thawing.

s Normal Donor Evaluation

To determine the enzyme activity range, 50 normal donors were run in duplicate. All samples were non-heat inactivated. This was done by one analyst over two plates. Individual healthy donors had enzyme activity ranges from 2.44 - 12.7 nmol/mL/hr.

Samples from MPS I patients receiving ERT and or gene therapy (e.g., ZFNs and IDUA transgene) were also evaluated as described in U.S. Provisional Application No. 62/802,568.

### Qualification Summary

The results of this qualification define the ability of this assay to detect the IDUA enzyme activity in human plasma. Assessment of the IDUA concentration curve showed reproducible accuracy and precision from the ULOQ of 5.0 ng/mL in-well (50 ng/mL in neat) to the LLOQ of 0.039 ng/mL in-well (0.39 ng/mL in neat). In addition, assessment of the 4MU concentration curve showed reproducible accuracy and precision from the ULOQ of 67.1 μM (corresponding mean enzyme activity 223.67 mnol/mL/hr) to the LLOQ of 0.197 μM (corresponding mean enzyme activity 0.66 nmol/mL/hr).

Inter-assay and intra-assay evaluation of IDUA controls indicates the assay was accurate and precise at five levels of drug concentrations (LLOQ, LQC, MQC, HQC, and ULOQ). The assay qualification data will be accepted, and for the purpose of controls, the IDUA concentrations determined during the qualification will be used for sample analysis: LQC=0.1 ng/mL in-well (1 ng/mL in neat), MQC=0.6 ng/mL in-well (6 ng/mL in neat), and HQC=4.0 ng/mL in-well (40 ng/mL in neat). The mean activity of the QCs was also determined during accuracy and precision assessment: LQC=3.37 nmol/mL/hr, MQC=21.8 nmol/mL/hr, and HQC=143 nmol/mL/hr. Moving forward, plate acceptance criteria will be set as follows: 4 of 6 QCs must have concentration and activity within ±20% of the QC values shown above and no two fail QCs can be from the same level.

To confirm reliability of the assay to measure samples that fall above the ULOQ, dilution linearity tests were performed. Heat-inactivated plasma samples spiked with a known concentration of IDUA were diluted at several levels and assayed. All quantifiable IDUA concentrations and activities of those diluted samples were within the acceptable range of precision and accuracy when back-calculated and compared to the theoretical concentration.

Selectivity of the assay was assessed by spiking 10 heat-inactivated individual plasma samples at LLOQ. When assayed concomitantly with the same unspiked individuals, all spiked samples yielded enzyme activity within target acceptance range and the unspiked samples were undetectable for enzyme activity. These results indicate that other components of the matrix do not affect the assay procedure.

The resistance to freeze-thaw cycle degradation of sample integrity was tested. Two individual samples underwent five freeze-thaw cycles. Each freeze-thaw cycle was assayed on the same plate. All freeze thaw cycles yielded enzyme activity within target acceptance range for IDUA activity as compared to samples subjected to one time freeze and thaw, indicating resistance to freeze-thaw affects for up to five cycles.

Finally, 50 normal donor plasma samples were evaluated for IDUA activity. Tested donors had enzyme activity ranges from 2.44-12.7 nmol/mL/hr.

### B. Leukocytes

IDUA assays as described above may also be conducted using leukocytes as the sample following essentially the same procedures.

Briefly, leukocytes are prepared from whole blood collected using either K2EDTA or sodium citrate blood collection tube and are sonicated in approximately 0.5-2 mL of water or water containing 1× protease inhibitor (Thermo) for 10 seconds while the tube is held in ice bath. Sonication is repeated twice for a total of 30 seconds. Leukocyte lysates are diluted at 1:1 ratio (MRD 2) with DPBS/0.2% BSA containing protease inhibitor (Sample Diluent). The sample is then mixed at a 1:1 ratio with 0.36 mM 4-Methylumbelliferyl α-L-iduronide substrate solution in a 96-well assay plate (20 μL of sample and 20 μL of substrate solution). Following an approximate 3-hour incubation at approximately 37° C., 160 μL of quenching solution is transferred to each well to stop the reaction. 100 μL of the samples are then transferred to a reading plate. The plate is then analyzed for fluorescent signal produced by free 4-MU. The activity of a sample is defined as the back-calculated value from the 4-MU curve with units nmol/mg/3 hrs (3 hrs because of the 3-hr incubation of sample with substrate). Concentration of leukocyte lysate is determined using BCA assay (Thermo) and use for activity calculation.

The IDUA assay contains two calibration curves prepared in sample diluent, an enzyme curve and a 4MU curve. The enzyme curve is used to measure the enzyme concentration and the 4MU curve is used to calculate enzyme activity in leukocytes, including in MPS I patients receiving ERT and/or gene therapy. During validation, 5 levels of quality control samples (ULOQ QC, HQC, MQC, LQC, and LLOQ QC) are included to define the quantifiable range of the assay. QCs can be prepared lysate from healthy donors (endogenous IDUA) or a combination of endogenous sample and recombinant hIDUA spiked into sample diluent. Three levels of quality control samples (HQC, MQC, LQC) are included in each run during sample testing with a minimum of one of the three QC samples being leukocyte lysate prepared from healthy donors. The same assay acceptance as used in plasma assay detailed above is used for leukocyte assay.

### Leukocyte Assay Reproducibility and Parallelism

Leukocyte pellets from 3 healthy donors were sonicated in 0.5 mL of water as described above. Each sample was diluted to MRD of 1:2 and 4 additional 2-fold serial dilutions with cold water. IDUA enzyme activity for each sample was measured using method described above. Enzyme activity for each sample was back-calculated using 4MU curve and the enzyme activity was normalized to the respective protein concentration.

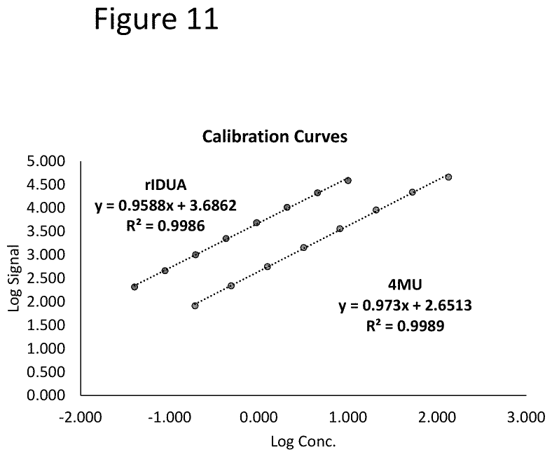

The resulting calibration curve is shown in FIG. 11. Further, the following Table shows enzymatic activity for the 3 donor leukocytes and CV.

| | Enzyme activity | | | % Bias from dilution 2 | | |
|---|---|---|---|---|---|---|
| | (nmol/3 hr/mg) | | | (MRD of 1:2) | | |
| Dilution | Donor 1 | Donor 2 | Donor 3 | Donor 1 | Donor 2 | Donor 3 |
| 2 | 67.3 | 31.4 | 31.6 | 0 | 0 | 0 |
| 4 | 65.7 | 38.3 | 36.2 | −2.4 | 22 | 14 |
| 8 | 66.9 | 39.3 | 38.3 | −0.6 | 25 | 21 |
| 16 | 65.2 | 38.2 | 37.0 | −3.1 | 22 | 17 |
| 32 | 69.5 | 36.6 | 36.5 | 3.2 | 17 | 15 |
| Overall | 66.9 | 36.7 | 35.9 | | | |
| % CV | 2.5 | 8.6 | 7.1 | | | |

% Bias = (sample activity − measured activity at MRD 1:2)/measured activity at MRD 1:2 * 100

Activity calculation=Back-calculated 4MU (nmol/mL)/3 hours/(protein concentration, mg/mL)

As shown, the overall CV for the measured enzyme activity is <20%. %Bias as compared to the MRD of 1:2 is within ±25%. This met the acceptance criteria for parallelism.

In addition, different sonication volumes were also evaluated for leukocyte samples. Different sonication volume was also explored to evaluate assay reproducibility and sonication condition. Three pellets from each donor was sonicated in either 0.25, 0.5, and 1 mL of water. Three donors were evaluated. All samples were analyzed at MRD of 1:2. IDUA enzyme activity was normalized to the respective protein concentration for each tested sample. The measured activity is consistent across with overall CV across different sonication volume less than 20%.

| | Enzyme activity, nmol/3 hr/mg | | |
|---|---|---|---|
| sonication volume | Donor 4 | Donor 5 | Donor 6 |
| 0.25 mL | 184.5 | 132.6 | 134.7 |
| 0.5 mL | 192.4 | 141.1 | 133.3 |
| 1 mL | 184.5 | 144.7 | 151.6 |
| Ave | 187.1 | 139.5 | 139.9 |
| % CV | 2.42 | 4.48 | 7.30 |

Assay precision was also evaluated using rIDUA spiked samples as well as endogenous IDUA in leukocytes.

Results are shown below.

Activity (nmol/3 hr/mL)

| | | | Endogenous IDUA | | |
|---|---|---|---|---|---|
| | rIDUA in | rIDUA in | in leukocyte lysate | rIDUA in | rIDUA in |
| | assay diluent | assay diluent | (diluted pooled healthy | assay diluent | assay diluent |
| | 10 ng/mL | 7.5 ng/mL | donor leukocyte lysate) | 0.2 ng/mL | 0.0655 ng/mL |
| | In-well | In-well | N/A | In-well | In-well |
| | ULOQ QC | HQC | MQC | LQC | LLOQ QC |
| N | 20 | 20 | 20 | 20 | 20 |
| Mean Activity | 206.35 | 169.15 | 33.27 | 6.68 | 2.74 |
| Intra assay % CV | 0.87 | 1.23 | 11.14 | 2.76 | 3.68 |
| Inter assay % CV | 5.13 | 4.96 | 14.42 | 6.87 | 7.41 |

As shown, overall intra and inter assay CV calculated using single factor ANOVA analysis was less than 20%.

Endogenous MQC performance was also evaluated across multiple runs and by at least 3 analysts. Results are shown below.

| Conc, | Activity (nmol/3 hr/mL) | | | | | | | |
|---|---|---|---|---|---|---|---|---|
| ng/mL | Analyst 1 | Analyst 1 | Analyst 2 | Analyst 3 | Analyst 1 | Analyst 1 | | |
| (in well) | Run 1 | Run 2 | Run 3 | Run 4 | Run 5 | Run 6 | Ave | % CV |
| 10.00 | 204.6 | 212.3 | 216.0 | 208.7 | 184.4 | 204.8 | 210.4 | 2.3 |
| 4.88 | 122.2 | 127.6 | 127.3 | 124.8 | 108.8 | 116.2 | 125.5 | 2.0 |
| 2.38 | 63.2 | 67.2 | 66.8 | 66.7 | 58.1 | 60.3 | 66.0 | 2.8 |
| 1.16 | 32.2 | 33.2 | 32.8 | 33.7 | 29.7 | 29.8 | 33.0 | 1.9 |
| 0.57 | 15.0 | 16.2 | 16.6 | 16.5 | 14.8 | 14.4 | 16.1 | 4.5 |

As shown, the overall CV across 3 analysts and 6 independent assays was less than 20% as well. rIDUA calibration curve back-calculated activity using 4MU calibration curve also shows acceptable overall performance with good parallelism between the two curves.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A system for measuring the levels and/or activity of iduronate-2-sulfatase (IDS) in a biological sample, the system comprising the following separate reaction mixtures:
(a) three or more separate reference standard reactions comprising a detectably-labeled IDS substrate comprising 4-methylumbelliferone-alpha-L-idopayranosiduronic Acid 2-Sufate Disodium salt (4MU-IDS), and recombinant IDS (rIDS), wherein the three or more reference standard reactions include different concentrations of rIDS;
(b) at least first, second and third separate quality control reactions comprising 4MU-IDS and rIDS, wherein the first quality control reaction comprises rIDS at a low quality control level (LQC), the second quality control reaction comprises rIDS at a medium quality control level (MQC) and the third quality control reaction comprises rIDS at a high quality control level (HQC)
(c) three or more separate substrate reactions comprising different concentrations of the detectably-labeled substrate; and
(d) a plurality of sample reactions comprising the biological sample and the detectably-labeled IDS substrate.

2. The system of claim 1, comprising duplicate reactions of at least the reference standards and quality control reactions.

3. The system of claim 1, wherein the at least first, second and third separate quality control reactions further comprise additional comprising quality control reactions with rIDS at the lower and/or upper levels of quantification and wherein the separate reaction mixtures of the system are included on the same matrix.

4. The system of claim 3, wherein for the three or substrate reactions comprise 4MU concentrations of 0.235 μM to 50 μM and further wherein the concentration of 4MU in the reference standard reactions comprise serial dilutions of a 1.25 to 2.5 mM stock 4MU solution.

5. A method of measuring the levels and/or activity of IDS in a biological sample, the method comprising the steps of:
(a) providing the system of separate reaction mixtures of claim 1;
(b) incubating the reactions;
(c) stopping the reactions of step (b) after a period of time;
(d) adding recombinant iduronidase (rIDUA) to each of the separate reactions;
(e) incubating the reactions of step (d);
(f) measuring the levels of detectable label from each reaction;
(g) generating (i) a reference standard curve from the levels of detectable label measured in the reference standard reactions and (ii) a substrate standard curve from the levels of detectable label measured in the substrate reactions;
(h) determining and/or quantifying the level and/or activity of IDS in the biological sample by measuring the levels of detectable label in the sample reactions and comparing the detected sample levels with the reference and substrate standard curves to determine enzyme activity in the sample.

6. The method of claim 7, further comprising determining an acceptable level criteria for the sample reaction measurements using one or more of the following parameters:
calculating the concentration of the standards, wherein at least 75% of the calculated concentrations for the standards must have a relative error (RE) within ±20% of low quality control (LQC), medium quality control (MQC) and high quality control (HQC);
calculating the concentration of the standards, wherein at least 75% of the calculated concentrations for the standards must have an RE within ±25% of the lower limit of quantification (LLOQ) or upper limit of quantification ULOQ;
substrate concentrations having a TE of ≤30% for LQC, MQC, HQC or ULOQ;
substrate concentrations having a TE of ≤40% for LLOQ;
% CVs of blank-corrected RFU for the reference and substrate standards is equal to or less than 20%; and/or
the substrate and/or reference curves have r²>0.98.

7. The method of claim 5, wherein the IDS standard curve as described herein providing the enzyme activity covers the range of quantification from at least 0.78 to 167 nmol/hr/mL.

8. The method of claim 5, wherein the sample is a plasma sample, a leukocyte sample, or a blood sample obtained from an MPS II subject.

9. The method of claim 8, wherein the MPS II subject has been treated with ERT and/or gene therapy reagents.

10. The method of claim 5, wherein the reactions of step (b) are incubated for 1-3 hours and the reactions of step (d) are incubated for 1 to 24 hours, further wherein the reactions are incubated at physiological temperature.

11. The method of claim 5, wherein the samples are contained in a micro plate and the levels of the detectable label are measured using a micro plate reader.

12. A system for measuring the levels and/or activity of IDUA in a biological sample, the system comprising the following separate reaction mixtures:
(a) three or more separate reference IDUA reactions comprising a detectably-labeled IDUA substrate comprising 4-methylumbelliferone-alpha-L-iduronide (4MU-IDUA) and recombinant IDS (rIDUA), wherein the three or more reference standard reactions include different concentrations of rIDUA;
(b) three or more separate substrate reactions comprising the detectably-labeled IDUA substrate
(c) at least first, second and third separate quality control reactions comprising 4MU-IDUA and rIDUA, wherein the first quality control reaction comprises rIDUA at a low quality control level, the second quality control reaction comprises rIDUA at a mid quality control level and the third quality control reaction comprises rIDUA at a high quality control level; and
(d) a plurality of sample reactions comprising the biological sample and the detectably-labeled IDUA substrate.

13. The system of claim 12, comprising duplicate reactions of at least the reference standards and quality control reactions.

14. The system of claim 12, the at least first, second and third separate quality control reactions further comprise additional comprising quality control reactions with rIDUA at the lower and/or upper levels of quantification and wherein the separate reaction mixtures of the system are included on the same matrix.

15. The system of claim 14, wherein for the three or substrate reactions comprise 4MU concentrations of 0.235 μM to 50 μM and further wherein the concentration of 4MU in the reference standard reactions comprise serial dilutions of a 1.25 to 2.5 mM stock 4MU solution.

16. A method of measuring the levels and/or activity of IDUA in a biological sample, the method comprising the steps of:
(a) providing the system of separate reaction mixtures of claim 12;
(b) incubating the reactions;
(c) measuring the levels of detectable label from each reaction;
(d) generating (i) a reference standard curve from the levels of detectable label measured in the reference standard reactions and (ii) a substrate standard curve from the levels of detectable label measured in the substrate reactions;
(e) determining and/or quantifying the level and/or activity of IDUA in the biological sample by measuring the levels of detectable label in the sample reactions and comparing the detected sample levels with the reference and substrate standard curves to determine enzyme activity in the sample.

17. The method of claim 16, further comprising determining an acceptable level criteria for the sample reaction measurements using one or more of the following parameters:
calculating the concentration of the standards, wherein at least 75% of the calculated concentrations for the standards must have an RE within ±20% of LQC, MQC and HQC;
calculating the concentration of the standards, wherein at least 75% of the calculated concentrations for the standards must have an RE within ±25% of the LLOQ or ULOQ;
substrate concentrations having a TE of ≤30% for LQC, MQC, HQC or ULOQ;
substrate concentrations having a TE of ≤40% for LLOQ;
% CVs of blank-corrected RFU for the reference and substrate standards is equal to or less than 20%; and/or
the substrate and/or reference curves have r²>0.98.

18. The method of claim 16, wherein the IDUA standard curve as described herein providing the enzyme activity covers the range of quantification from at least 0.66 to 167 nmol/hr/mL.

19. The method of claim 16, wherein the sample is a plasma sample, a leukocyte sample or a blood sample obtained from an MPS I subject.

20. The method of claim 19, wherein the MPS I subject has been treated with ERT and/or gene therapy reagents.

21. The method of claim 16, wherein the reactions of step (b) are incubated for 1-3 hours, further wherein the reactions are incubated at physiological temperature.

22. The method of claim 16, wherein the samples are contained in a micro plate and the levels of the detectable label are measured using a micro plate reader.
